SEQUENCE LISTING (103 sequences)

SEQ ID NO: 1 - protein, 143 aa, Bacillus subtilis
MLEFDTIKDSKQLNGLALAYIGDAIFEVYVRHHLLKQGFTKPNDLHKKSSRIVSAKSQAEILFFLQNQSFFTEEEEAVLKRGRNAKSGTTPKNTDVQTYRYSTAFEALLGYLFLEKKEERLSQLVAEAIQFGTSGRKTNESAT

SEQ ID NO: 2 - protein, 132 aa, Caldicellulosiruptor kristjanssonii
MLSPLVYAYIGDAVYELFVRNKIIAENPDLTPYLYYLRTTMYVKASSQAMAIKKLYEELDEDEKRIVKRGRNAKPKTIPRNAKLSDYKYATALEALIGYLYLANNIERLNYILSQTYDIITEEYSNAKNSCQ

SEQ ID NO: 3 - DNA, 399 bp, Caldicellulosiruptor kristjanssonii
atgcttagtcctttagtatatgcttatattggagatgcagtatatgagttgtttgtaagaaacaaaataatagctgaaaatccagatttgaccccctacctatactatcttagaactactatgtatgtaaaagcttcgagtcaagcaatggctataaaaaaattatatgaagagcttgatgaagatgaaaaaagaattgtaaagagaggcagaaatgcaaaaccaaaaaccattcccagaaatgccaagttgagtgattataaatatgccacggcccttgaggcactaattggttatctttatttagcaaataacattgagagattaaattatattctttcacaaacgtatgatataataactgaagaatacagcaatgccaagaatagctgtcaataa

SEQ ID NO: 4 - protein, 125 aa, Clostridium ramosum
MGPELINASVLAYLGDSIFEVLVRDYLVKESGFVKPNDLQREAVKYVSASSHAAFMHDMLDEEFFSADEVGTYKRGRNTKGSKNESLDHMHSTGFEAVIGTLYLEENFDRIKVIFERYKQYINNK

SEQ ID NO: 5 - DNA, 378 bp, Clostridium ramosum
atgggccctgaactgattaatgcaagcgttctggcatatctgggtgatagcatttttgaagttctggtgcgtgattatctggtgaaagaaagcggttttgtgaaaccgaatgatctgcagcgtgaagccgttaaatatgttagcgcaagcagccatgcagcatttatgcatgatatgctggatgaagaatttttcagcgcagatgaagttggcacctataaacgtggtcgtaataccaaaggtagcaaaaatgaaagcctggatcatatgcatagcaccggttttgaagcagttattggcaccctgtatctggaagaaaatttcgatcgcatcaaagtgatcttcgagcgctataaacagtacatcaacaacaaataa

SEQ ID NO: 6 - protein, 140 aa, Clostridium thermocellum
MVWEFFDKITGEFNYKPDEVSQLSPLVLAYIGDAVYEVFIRTMLVSGGNVPVHVLHKRSIAYVKAKAQSDIVHRIMPLLTEEELNIVRRGRNAKSATVPKNADITDYRYATGFESLLGFLYLKKDYDRLMDILRMAVSQN

SEQ ID NO: 7 - DNA, 423 bp, Clostridium thermocellum
atggtttgggaattttttgacaaaattacaggtgagtttaattacaaaccggatgaagtaagccaactgtcgcctttagtgcttgcatacataggtgacgccgtgtatgaggttttcatccgtacaatgcttgtgtccggaggaaacgtaccggtacatgttctccataagcgctccattgcttatgtcaaagcaaaggcacagtcggatattgtccacaggataatgcctttgctgacggaggaggagcttaatattgtccgcaggggaaggaacgccaaatcggccacggttccgaaaaatgcggatattacggattacaggtatgctaccggttttgagtctttgttgggttttctttatttgaaaaaagattatgaccgattgatggatatattgcgaatggctgtttcacagaattga

SEQ ID NO: 8 - protein, 134 aa, Faecalibacterium prausnitzii
MNESEKIDPRELSPLALAFVGDSVLELLVRQRLVEHHRLSAGKLNAEKVKYVSAKAQFREEQLLEPLFTEDELAVFKRGRNASKASVAKHASPEEYRASTGFECLLGWLYLNGQLERVHQLFEVLWQQFDPDQK

SEQ ID NO: 9 - DNA, 405 bp, Faecalibacterium prausnitzii
atgaatgaaagcgaaaaaattgatccgcgtgaactgagtccgctggcactggcatttgttggtgatagcgttctggaactgctggttcgtcagcgtctggttgaacatcatcgtctgagcgcaggtaaactgaatgcagaaaaagttaaatacgttagcgccaaagcacagtttcgtgaagaacagctgctggaaccgctgtttaccgaagatgaactggcagtttttaaacgtggtcgtaatgcaagcaaagcaagcgttgcaaaacatgcaagtccggaagaatatcgtgcaagcaccggttttgaatgtctgctgggttggctgtatctgaatggtcagctggaacgtgttcatcagctgtttgaagttctgtggcagcagtttgatcctgatcagaaataa

SEQ ID NO: 10 - protein, 129 aa, Fusobacterium nucleatum subsp. nucleatum
MDNVDFSKDIRDYSGLELAFLGDAIWELEIRKYYLQFGYNIPTLNKYVKAKVNAKYQSLIYKKIINDLDEEFKVIGKRAKNSNIKTFPRSCTVMEYKEATALEAIIGAMYLLKKEEEIKKIINIVIKGE

SEQ ID NO: 11 - DNA, 390 bp, Fusobacterium nucleatum subsp. nucleatum
atggacaatgtagatttttcaaaggatataagagattacagtggactggaattagcatttttaggagatgctatttgggaactggaaataagaaaatattacttacaatttggctataatattcctactttaaataaatatgttaaagctaaggtaaatgcaaaatatcaaagtctgatttataagaaaattataaatgatttagatgaagaatttaaagttataggaaaaagagctaaaaatagtaacataaaaacttttccaaggagttgtacagtgatggaatataaggaagcgacagccttagaagctattatcggagcaatgtatttgttaaaaaaagaagaagaaataaaaaaaattataaatatagttataaagggagaatga

SEQ ID NO: 12 - protein, 132 aa, Staphylococcus epidermidis
MAKHMNVKLLNPLTLAYMGDAVLDQHVREYIVLKLQSKPHRLHQVSKSYVSAKSQAKTLEYLLDIDWFTEEELSVLKRGRNAKSYTKAKNTDIQTYRKSSALEAVIGFLYLDHQSERLENLLETIVRIVDER

SEQ ID NO: 13 - DNA, 399 bp, Staphylococcus epidermidis
atggctaaacatatgaacgtaaaacttcttaatcctttaacattggcatatatgggtgatgcagtacttgatcaacatgtgcgtgaatatatcgtgctaaaattacaaagtaaacctcatcgtttgcaccaagtatcgaaaagttacgtttcagcgaaaagtcaagctaagactttagagtatttgttagatattgactggtttacagaggaagagctaagtgttttaaaacgaggacgtaacgctaaaagttatacaaaagctaaaaatactgacattcaaacttatcgtaaaagttcagcgttagaagctgttatcggatttttatatttagaccatcaatcagaacgattagaaaacttattagaaacaattgttaggatagtggatgaaaggtaa

SEQ ID NO: 14 - protein, 140 aa, Thermotoga maritima
MEKLFRFEAEPEKLPPAVLAYLGDAVLELIFRSRFTGDYRMSVIHERVKEHTSKHGQAWMLENIWNLLDEREQEIVKRAMNSKAAKRHGNDPTYRKSTGFEALIGYLFLKREFDRIEELLRVVMDLESLRKKNPGGSAQE

SEQ ID NO: 15 - DNA, 423 bp, Thermotoga maritima
atggaaaaactcttcagattcgaagcagaaccggagaaactgccaccggccgttctagcgtatctgggagatgccgttctggagctcatcttcagatcgagattcacaggagattacagaatgtccgtcatacacgagagggtcaaggaacacacctcgaaacacggtcaggcatggatgctggagaatatatggaatctcctcgacgaaagagagcaagaaatagttaaaagagcgatgaattcgaaggcagcgaaaagacacgggaacgaccctacatacagaaagagcaccggtttcgaagctttgatcgggtatctattcttgaaaagagaattcgacagaattgaagaactgcttcgggtggtgatggatcttgagagtctacggaagaaaaatcctggaggaagcgctcaggaataa

SEQ ID NO: 16 - protein, 136 aa, Thermoanaerobacter tengcongensis
MEKDKMILVKEKGVLDLSPLVLAFIGDAVYSLYVRTKIVEKGNMKLAHLNEQTVKYVKASSQARSLERIYDLLTEEEKEIVRRGRNAKASTVPKGASVKEYKYATAFEALVGYLYLLERFDRLYFLLSLSMEYTEE

SEQ ID NO: 17 - DNA, 477 bp, Thermoanaerobacter tengcongensis
atgggcagcagccatcatcatcatcatcacagcagcggcctggaagttctgttccaggggccccatatggaaaaggataagatgattcttgtaaaggaaaagggggttttagacttatccccccttgttttggctttcattggagatgcggtttacagcctttatgtcagaactaagattgtggagaaagggaatatgaaattggctcatttaaatgagcaaactgtgaagtacgttaaggcatcttcacaggctaggtctcttgagcgaatttacgaccttctcactgaagaagaaaaggaaattgtgagaaggggaagaaatgccaaagcttctacagttccaaaaggagcaagtgttaaagagtataagtatgccactgcctttgaagcattagtgggatatttgtaccttttagaaagatttgataggctttactttcttttgagcctttctatggaatacacggaagaatga

SEQ ID NO: 18 - protein, 140 aa, artificial
  AA sequence of chimeric protein Ct(FpH)
MVWEFFDKITGEFNYKPDEVSQLSPLVLAYIGDAVYEVFIRTMLVSGGNVPVHVLNAEKVKYVKAKAQSDIVHRIMPLLTEEELNIVRRGRNAKSATVPKNADITDYRYATGFESLLGFLYLKKDYDRLMDILRMAVSQN

SEQ ID NO: 19 - DNA, 423 bp, artificial
  NA sequence of chimeric protein Ct(FpH)
atggtttgggaattttttgacaaaattacaggtgagtttaattacaaaccggatgaagtaagccaactgtcgcctttagtgcttgcatacataggtgacgccgtgtatgaggttttcatccgtacaatgcttgtgtccggaggaaacgtaccggtacatgttctgaatgcagaaaaagttaaatatgtcaaagcaaaggcacagtcggatattgtccacaggataatgcctttgctgacggaggaggagcttaatattgtccgcaggggaaggaacgccaaatcggccacggttccgaaaaatgcggatattacggattacaggtatgctaccggttttgagtctttgttgggttttctttatttgaaaaaagattatgaccgattgatggatatattgcgaatggctgtttcacagaattaa

SEQ ID NO: 20 - protein, 140 aa, artificial
  AA sequence of chimeric protein Ct(FpL)
MVWEFFDKITGEFNYKPDEVSQLSPLVLAYIGDAVYEVFIRTMLVSGGNVPVHVLHKRSIAYVKAKAQSDIVHRIMPLLTEEELNIVRRGRNASKASVAKHASPEEYRYATGFESLLGFLYLKKDYDRLMDILRMAVSQN

SEQ ID NO: 21 - DNA, 423 bp, artificial
  NA sequence of chimeric protein Ct(FpL)
atggtttgggaattttttgacaaaattacaggtgagtttaattacaaaccggatgaagtaagccaactgtcgcctttagtgcttgcatacataggtgacgccgtgtatgaggttttcatccgtacaatgcttgtgtccggaggaaacgtaccggtacatgttctccataagcgctccattgcttatgtcaaagcaaaggcacagtcggatattgtccacaggataatgcctttgctgacggaggaggagcttaatattgtccgcaggggaaggaacgcgtcaaaagcaagcgttgcaaaacatgcaagtccggaagaatacaggtatgctaccggttttgagtctttgttgggttttctttatttgaaaaaagattatgaccgattgatggatatattgcgaatggctgtttcacagaattaa

SEQ ID NO: 22 - protein, 140 aa, artificial
  AA sequence of chimeric protein Ct(FpHL)
MVWEFFDKITGEFNYKPDEVSQLSPLVLAYIGDAVYEVFIRTMLVSGGNVPVHVLNAEKVKYVKAKAQSDIVHRIMPLLTEEELNIVRRGRNASKASVAKHASPEEYRYATGFESLLGFLYLKKDYDRLMDILRMAVSQN

SEQ ID NO: 23 - DNA, 422 bp, artificial
  NA sequence of chimeric protein
atggtttgggaattttttgacaaaattacaggtgagtttaattacaaaccggatgaagtaagccaactgtcgcctttagtgcttgcatacataggtgacgccgtgtatgaggttttcatccgtacaatgcttgtgtccggaggaaacgtaccggtacatgttctgaatgcagaaaaagttaaatatgtcaaagcaaaggcacagtcggatattgtccacaggataatgcctttgctgacggaggaggagcttaatattgtccgcaggggaaggaacgcgtcaaaagcaagcgttgcaaaacatgcaagtccggaagaatacaggtatgctaccggttttgagtctttgttgggttttctttatttgaaaaaagattatgaccgattgatggatatattgcgaatggctgtttcacagaatta

SEQ ID NO: 24 - protein, 141 aa, artificial
  AA sequence of chimeric protein Bs(FpH)
MVEFDTIKDSKQLNGLALAYIGDAIFEVYVRHHLLKQGFTKPNDLNAEKYVSAKSQAEILFFLQNQSFFTEEEEAVLKRGRNAKSGTTPKNTDVQTYRYSTAFEALLGYLFLEKKEERLSQLVAEAIQFGTSGRKTNESAT

SEQ ID NO: 25 - DNA, 426 bp, artificial
  NA sequence of chimeric protein Bs(FpH)
atggttgaatttgatacgataaaagattctaagcagcttaacggtcttgcgcttgcttatataggtgatgccatttttgaagtgtatgtcaggcatcacctgcttaagcagggctttaccaaaccaaatgatctgaatgcagaaaaatatgtttcagcaaagtcacaggctgagatcctattttttctgcagaatcaatcattttttacggaagaagaggaagcggtgctgaaaagaggcagaaatgccaagtcagggacaacacctaaaaatacagatgttcagacgtaccgctacagtacagcatttgaagcgcttctgggctacctttttctagagaaaaaagaggaacgacttagtcagctcgtagccgaagctatacaattcgggacgtcagggaggaaaacaaatgagtcagcaacataa

SEQ ID NO: 26 - protein, 143 aa, artificial
  AA sequence of chimeric protein Bs(FpL)
MVEFDTIKDSKQLNGLALAYIGDAIFEVYVRHHLLKQGFTKPNDLHKKSSRIVSAKSQAEILFFLQNQSFFTEEEEAVLKRGRNASKASVAKHASPEEYRYSTAFEALLGYLFLEKKEERLSQLVAEAIQFGTSGRKTNESAT

SEQ ID NO: 27 - DNA, 432 bp, artificial
  NA sequence of chimeric protein Bs(FpL)
atggttgaatttgatacgataaaagattctaagcagcttaacggtcttgcgcttgcttatataggtgatgccatttttgaagtgtatgtcaggcatcacctgcttaagcagggctttaccaaaccaaatgatcttcataagaaatcaagccggattgtttcagcaaagtcacaggctgagatcctattttttctgcagaatcaatcattttttacggaagaagaggaagcggtgctgaaaagaggcagaaacgcgtcaaaagcaagcgttgcaaaacatgcaagtccggaagaataccgctacagtacagcatttgaagcgcttctgggctacctttttctagagaaaaaagaggaacgacttagtcagctcgtagccgaagctatacaattcgggacgtcagggaggaaaacaaatgagtcagcaacataa

SEQ ID NO: 28 - protein, 131 aa, artificial
  AA sequence of chimeric protein Se(FpH)
MVAKHMNVKLLNPLTLAYMGDAVLDQHVREYIVLKLQSKPHRLNAEKYVSAKSQAKTLEYLLDIDWFTEEELSVLKRGRNAKSYTKAKNTDIQTYRKSSALEAVIGFLYLDHQSERLENLLETIVRIVDER

SEQ ID NO: 29 - DNA, 394 bp, artificial
  NA sequence of chimeric protein Se(FpH)
atggtggctaaacatatgaacgtaaaacttcttaatcctttaacattggcatatatgggtgatgcagtacttgatcaacatgtgcgtgaatatatcgtgctaaaattacaaagtaaacctcatcgtctgaatgcagaaaaatacgtttcagcgaaaagtcaagctaagactttagagtatttgttagatattgactggtttacagaggaagagctaagtgttttaaaacgaggacgtaacgctaaaagttatacaaaagctaaaaatactgacattcaaacttatcgtaaaagttcagcgttagaagctgttatcggatttttatatttagaccatcaatcagaacgattagaaaacttattagaaacaattgttaggatagtggatgaaataa

SEQ ID NO: 30 - DNA, 27 bp, artificial
  Starter FckminiIII
cctccatggtcagtcctttagtatatg

SEQ ID NO: 31 - DNA, 30 bp, artificial
  Starter RckminiIII
cctctcgagttattgacagctattcttggc

SEQ ID NO: 32 - DNA, 28 bp, artificial
  Starter FcrminiIII
ggaccatgggccctgaactgattaatgc

SEQ ID NO: 33 - DNA, 30 bp, artificial
  Starter RcrminiIII
ggcctcgagttatttgttgttgatgtactg

SEQ ID NO: 34 - DNA, 28 bp, artificial
  Starter FctminiIII
caggcatatggtttgggaattttttgac

SEQ ID NO: 35 - DNA, 28 bp, artificial
  Starter RctminiIII
gacctcgagtcaattctgtgaaacagcc

SEQ ID NO: 36 - DNA, 27 bp, Artificial
  Starter FfpminiIII
ggaccatggacgaaagcgaaaaaattg

SEQ ID NO: 37 - DNA, 31 bp, Artificial
  Starter RfpminiIII
gcgctcgagttatttctgatcaggatcaaac

SEQ ID NO: 38 - DNA, 30 bp, artificial
  Starter FfnminiIII
ccgcatatggacaatgtagatttttcaaag

SEQ ID NO: 39 - DNA, 54 bp, artificial
  Starter RfnminiIII
gtgctcgagtcatcattctccctttataactatatttataattttttttatttc

SEQ ID NO: 40 - DNA, 31 bp, artificial
  Starter FseminiIII
tagacatatggcagtggctaaacatatgaac

SEQ ID NO: 41 - DNA, 25 bp, artificial
  Starter RseminiIII
atctcgagctacctttcatccacta

SEQ ID NO: 42 - DNA, 29 bp, artificial
  Starter FtmminiIII
gcttcatatggaaaaactcttcagattcg

SEQ ID NO: 43 - DNA, 27 bp, artificial
  Starter RtmminiIII
cttctcgagttattcctgagcgcttcc

SEQ ID NO: 44 - DNA, 32 bp, artificial
  Starter FttminiIII
cgcacatatggaaaaggataagatgattcttg

SEQ ID NO: 45 - DNA, 32 bp, artificial
  Starter RttminiIII
gctctcgagtcattcttccgtgtattccatag

SEQ ID NO: 46 - DNA, 36 bp, artificial
  Starter UniShPreA
agatcggaagagcgtcgtgtagggaaagagtgtaga

SEQ ID NO: 47 - DNA, 23 bp, artificial
  Starter UniShRT
tctacactctttccctacacgac

SEQ ID NO: 48 - DNA, 33 bp, artificial
  Starter PreA3Univ
gatcggaagagcacacgtctgaactccagtcac

SEQ ID NO: 49 - DNA, 34 bp, artificial
  Starter 910fT7
taatacgactcactatagggctgctcgcgcgttg

SEQ ID NO: 50 - DNA, 31 bp, artificial
  Starter 910rP6
ggaaaaaaatcagacacaactgacgcgatcg

SEQ ID NO: 51 - DNA, 41 bp, artificial
  Starter 949fT7
taatacgactcactatagggcctctctctctggccacgatc

SEQ ID NO: 52 - DNA, 31 bp, artificial
  Starter 949rP6
ggaaaaaaatgccctgtacagcaggcataag

SEQ ID NO: 53 - DNA, 39 bp, artificial
  Starter 2021fT7
taatacgactcactatagggctcctatcatggccgttgc

SEQ ID NO: 54 - DNA, 30 bp, artificial
  Starter 2021rP6
ggaaaaaaacttcgagatcagggttggacg

SEQ ID NO: 55 - DNA, 42 bp, artificial
  Starter 3292fT7
taatacgactcactatagggtaccgcgatcaacactgtcgtc

SEQ ID NO: 56 - DNA, 29 bp, artificial
  Starter 3292rP6
ggaaaaaaacgaatcaggacgtctggacg

SEQ ID NO: 57 - DNA, 41 bp, artificial
  Starter 4486fT7
taatacgactcactatagggctgtctcccctcggtttcatc

SEQ ID NO: 58 - DNA, 28 bp, artificial
  Starter 4486rP6
ggaaaaaaatcgacagacgacagcgctg

SEQ ID NO: 59 - DNA, 42 bp, artificial
  Starter 4754fT7
taatacgactcactatagggctcatcgcctcgatgaaccaag

SEQ ID NO: 60 - DNA, 29 bp, artificial
  Starter 4754rP6
ggaaaaaaactactgctttcgagcggtcg

SEQ ID NO: 61 - DNA, 21 bp, artificial
  Starter WSSWf
sswctctctctggccacgatc

SEQ ID NO: 62 - DNA, 16 bp, artificial
  Starter WSSWr
wttccctcccagcacg

SEQ ID NO: 63 - DNA, 21 bp, artificial
  Starter ANNTf
  misc_feature (1)..(2): n is a, c, g, or t
nntctctctctggccacgatc

SEQ ID NO: 64 - DNA, 16 bp, artificial
  Starter ANNTr
tttccctcccagcacg

SEQ ID NO: 65 - DNA, 21 bp, artificial
  Starter NCCNf
  misc_feature (3)..(3): n is a, c, g, or t
ccnctctctctggccacgatc

SEQ ID NO: 66 - DNA, 16 bp, artificial
  Starter NCCNr
  misc_feature (1)..(1): n is a, c, g, or t
nttccctcccagcacg

SEQ ID NO: 67 - DNA, 24 bp, artificial
  Starter 3T-r
tttacctcccagcacgaccgcgac

SEQ ID NO: 68 - DNA, 26 bp, artificial
  Starter 3T-f
cctctctctctggccacgatcgcgtc

SEQ ID NO: 69 - DNA, 21 bp, artificial
  Starter 4C-r
gccctcccagcacgaccgcga

SEQ ID NO: 70 - DNA, 22 bp, artificial
  Starter 4G-r
cccctcccagcacgaccgcgac

SEQ ID NO: 71 - DNA, 19 bp, artificial
  Starter 4T-r
accctcccagcacgaccgc

SEQ ID NO: 72 - DNA, 28 bp, artificial
  Starter 4-f
aacctctctctctggccacgatcgcgtc

SEQ ID NO: 73 - DNA, 28 bp, artificial
  Starter 11C-r
cctccctctctggccacgatcgcgtcag

SEQ ID NO: 74 - DNA, 28 bp, artificial
  Starter 11G-r
cctcgctctctggccacgatcgcgtcag

SEQ ID NO: 75 - DNA, 28 bp, artificial
  Starter 12A-r
cctctatctctggccacgatcgcgtcag

SEQ ID NO: 76 - DNA, 24 bp, artificial
  Starter 11/12-f
tttccctcccagcacgaccgcgac

SEQ ID NO: 77 - DNA, 22 bp, artificial
  Starer CtR1Up
gcttcataagcgctccattgct

SEQ ID NO: 78 - DNA, 22 bp, artificial
  Starter CtR1Dw
agcaatggagcgcttatgaagc

SEQ ID NO: 79 - DNA, 32 bp, artificial
  Starter CtR2Up
caatgccaaatcggccacggttccgaaaaatg

SEQ ID NO: 80 - DNA, 27 bp, artificial
  Starter CtR2Dw
atccgtaatatccgcatttttcggaac

SEQ ID NO: 81 - DNA, 17 bp, artificial
  Starter FpR1Up
atgcagaaaaagttaaa

SEQ ID NO: 82 - DNA, 17 bp, artificial
  Starter FpR1Dw
tttaactttttctgcat

SEQ ID NO: 83 - DNA, 27 bp, artificial
  Starter FpR2Up
cgtcaaaagcaagcgttgcaaaacatg

SEQ ID NO: 84 - DNA, 27 bp, artificial
  Starter FpR2Dw
ttcttccggacttgcatgttttgcaac

SEQ ID NO: 85 - DNA, 19 bp, artificial
  Starter CpR1f
tatgtcaaagcaaaggcac

SEQ ID NO: 86 - DNA, 20 bp, artificial
  Starter CpR1r
tcagaacatgtaccggtacg

SEQ ID NO: 87 - DNA, 30 bp, artificial
  Starter CpR2f
tacaggtatgctaccggttttgagtctttg

SEQ ID NO: 88 - DNA, 19 bp, artificial
  Starter
cgttccttcccctgcggac

SEQ ID NO: 89 - DNA, 16 bp, artificial
  Starter FpR1f
tacgttagcgccaaag

SEQ ID NO: 90 - DNA, 16 bp, artificial
  Starer FpR1r
ttacctgcgctcagac

SEQ ID NO: 91 - DNA, 22 bp, artificial
  Starter FpR2f
tatcgtgcaagcaccggttttg

SEQ ID NO: 92 - DNA, 26 bp, artificial
  Starter FpR2r
cgaccacgtttaaaaactgccagttc

SEQ ID NO: 93 - DNA, 20 bp, artificial
  Starter BsR1f
tatgtttcagcaaagtcaca

SEQ ID NO: 94 - DNA, 23 bp, artificial
  Starter BsR1r
tcagatcatttggtttggtaaag

SEQ ID NO: 95 - DNA, 17 bp, artificial
  Starter BsR2f
taccgctacagtacagc

SEQ ID NO: 96 - DNA, 20 bp, artificial
  Starter BsR2r
cgtttctgcctcttttcagc

SEQ ID NO: 97 - DNA, 19 bp, artificial
  Starter SeR1f
tacgtttcagcgaaaagtc

SEQ ID NO: 98 - DNA, 31 bp, artificial
  Starter SeR1rf
tcagacgatgaggtttactttgtaattttag

SEQ ID NO: 99 - DNA, 22 bp, artificial
  Starter SeR2f
tatcgtaaaagttcagcgttag

SEQ ID NO: 100 - DNA, 22 bp, artificial
  Starter SeR2r
cgttacgtcctcgttttaaaac

SEQ ID NO: 101 - DNA, 18 bp, artificial
  Starter ET-long
gtccggcgtagaggatcg

SEQ ID NO: 102 - DNA, 16 bp, artificial
  Starter ET-reverse
tcccattcgccaatcc

SEQ ID NO: 103 - RNA, 14 nt, phi6 bacteriophage
gggaaaccucucuc

(19) United States
(12) Patent Application Publication
(10) Pub. No.: US 2019/0032035 A1
(43) Pub. Date: Jan. 31, 2019

(54) Mini-III RNases, Methods for Changing the Specificity of RNA Sequence Cleavage by Mini-III RNases, and Uses Thereof

(71) Applicant: Biotech Innovations Sp. z o.o., Warszawa (PL)

(72) Inventors: Janusz Bujnicki, Warszawa (PL); Krzysztof Skowronek, Izabelin (PL); Dawid Glów, Konskowola (PL); Malgorzata Kurkowska, Warszawa (PL)

(21) Appl. No.: 16/071,867

(22) PCT Filed: Jul. 20, 2017
(86) PCT No.: PCT/IB2017/050296

§ 371 Date: Jul. 20, 2018

(30) Foreign Application Priority Data
Jan. 22, 2016 (PL) PL415883

(51) Int. Cl. C12N 9/22; C12N 15/52

(52) U.S. Cl. CPC C12N 9/22; C12Y 301/26003; C12N 15/52

(57) ABSTRACT

The object of the invention is a Mini-III RNase with amino acid sequence comprising an acceptor part, and a transplantable a4 helix, and a transplantable a5b-a6 loop, which form structures of a4 helix and a5b-a6 loop, respectively, in the Mini-III RNase structure, wherein the fragments which form structures of a4 helix and a5b-a6 loop, respectively, correspond structurally to respective structures of a4 helix and a5b-a6 loop formed by amino acid sequence fragments 46-52 and 85-98, respectively, of Mini-III RNase from Bacillus stubtilis shown in SEQ ID NO: 1, wherein the said Mini-III RNase exhibits sequence specificity in dsRNA cleavage being dependent only on a ribonucleotide sequence of the substrate, and independent from an occurrence of secondary structures in the substrate's structure, and independent from a presence of other assisting proteins, and wherein the Mini-III RNase is not the Mini-III protein from Bacillus stubtilis of SEQ ID NO: 1, nor SEQ ID NO: 1 with D94R mutation. The invention also relates to a method of obtaining a chimeric Mini-III RNase, a Mini-III RNase encoding construct, a cell with a Mini-III RNase encoding gene, use of Mini-III RNase for dsRNA cleavage, as well as a method of dsRNA cleavage depending only on a ribonucleotide sequence.

### TECHNICAL FIELD

The object of the invention are novel natural and chimeric Mini-III RNases with amino acid sequences comprising an acceptor part and part of a transplantable α4 helix and a transplantable α5b-α6 loop which form the structures of α4 helix and α5b-α6 loop, respectively, in the Mini-III RNase structure, wherein Mini-III RNases show sequence specificity in dsRNA cleavage being dependent only on a ribonucleotide sequence of a substrate and independent from an occurrence of secondary structures in the substrate's structure, and independent of a presence of other assisting proteins, and wherein the Mini-III RNase is not a Mini-III protein from Bacillus stubtilis of SEQ ID NO: 1, nor of SEQ ID NO: 1 with D94R mutation. The invention also relates to a method of obtaining chimeric Mini-III RNase, a Mini-III RNase encoding construct, a cell with a Mini-III RNase encoding gene, a use of Mini-III RNase for dsRNA cleavage, as well as a method of dsRNA cleavage depending only on a ribonucleotide sequence.

### BACKGROUND ART

One of the basic tools of molecular biology are proteins with a clearly defined activity, used, for example, in genetic engineering, diagnostics, medicine and industry for manufacturing and processing various products. One example of such enzymes are DNA endonucleases, also referred to as restriction enzymes, which recognize and cleave specific sequences of double-stranded DNA (dsDNA).

Ribonucleases (RNases) are counterparts of DNA restriction enzymes, and they play an important role in the processing and degradation of RNA in cells by taking part in various biochemical reactions based on exo- or endonucleolytic cleavage of RNA molecules. Exoribonucleases degrade

RNA molecules in a sequence-independent manner, starting from their ends, while endoribonucleases (endoRNases) cleave single- or double-stranded RNA molecules (ssRNA and dsRNA, respectively) inside. Although many RNases show substrate specificity, their target sequence is usually limited to one or few nucleotides in ssRNA, very often in a context of a particular secondary and tertiary structure of the whole molecule. One of such enzymes is the phage protein RegB, which cleaves the GGAG sequence in the middle. To cleave efficiently, RegB requires additional determinants, such as a proper RNA secondary structure and enzyme interaction with a ribosomal protein S1 (Lebars, I., et al., J Biol Chem, (2001) 276, 13264-13272, and Saida, F., et al., (2003) Nucleic Acids Res, 31, 2751-2758.).

There are other sequence-specific ribonucleases known, but they are unsuitable for engineering due to the fact that, in addition to a suitable nucleotide sequence, a unique structure formed by a particular ssRNA fragment is required. There have been attempts to change the substrate specificity of T1 and MC1 RNases (Hoschler, K., et al., J Mol Biol, (1999) 294, 1231-1238, Numata, T., et al., Biochemistry, (2003) 42, 5270-5278). In these two cases, enzyme variants with specificity narrowed from single nucleotide to two nucleotides were produced (Numata, T., et al., Biochemistry, (2003) 42, 5270-5278; Czaja, R., et al., Biochemistry, (2004) 43, 2854-2862; Struhalla, M., et al., Chembiochem, (2004) 5, 200-205). However, low or no specificity towards hydrolysed RNA sequence greatly limits its processing, and hence also the possibility to apply such enzymes.

Apart from proteins, hammerhead ribozymes, catalytic DNA molecules (DNAzymes), and artificial enzymes based on peptide nucleic acids (PNAzymes) are also used to cleave RNA sequences. Described molecules may be designed to obtain sequence-specific cleavage of RNA molecules. Hammerhead ribozymes are 30-nucleotide RNA molecules initially discovered in plant viruses (Prody, Ga., et al., Science, (1986) 231, 1577-1580). They form three stems, wherein the sequences forming stems I and III bind complementary sequences of substrate RNA molecule flanking the target sequence UH (where H is any nucleotide except G), which is cleaved in the middle. Hammerhead ribozymes may be designed to cleave target sequences in cis or trans conformation (Usman, N., et al., Curr. Opin. Struct. Biol. (1996) 4, 527-533). DNAzymes are catalytic DNA molecules that have been identified using in vitro selection from random DNA sequences. Thus far, many DNAzymes with a broad range of specificity have been described. Designed Cu²⁺ dependent PNAzymes also provide a possibility for highly selective cleavage of RNA molecules. They are designed to bind with an RNA complementary sequence and form a bulge of four nucleotides in a target molecule. If the formed bulge comprises the AYRA sequence (where Y=C, U; R=A, G), it can be cleaved by the PNAzyme (Murtola M., et al., J Am Chem Soc (2010) 26, 8984-8990).

Enzymes with the ability to process dsRNA belong to ribonuclease III superfamily in which four families have been identified: Dicer, Drosha, RNase III, and Mini-III. All proteins classified thereinto are characterized by the catalytic domain of ribonuclease III. Ribonuclease Mini-III from Bacillus stubtilis has a domain of this type, yet it does not have a dsRNA binding domain typical for other known members of ribonuclease III superfamily. Genes encoding Mini-III are present in genomes of Gram-negative bacteria and in plant plastids. In both bacteria and plastids, the Mini-III enzyme is involved in the process of 23S rRNA maturation. A natural substrate for this protein is 23S pre-rRNA in which 3′ and 5′ ends are cleaved. The sequence cleaved by Mini-III in the natural substrate of 23S pre-rRNA has been determined, and it has been found that close to the cleavage site the 23S pre-rRNA fragment attains a partly double-stranded and partly irregular structure with unpaired single-stranded elements. The studies conducted thus far have suggested that the Mini-III may have predispositions to recognize irregularities in the dsRNA helix structure (Redko, Y., et al., Molecular Microbiology, (2008) 68(5), 1096-1106). The activity of Mini-III towards 23S pre-rRNA is significantly stimulated by L3 protein which acts on the substrate, and not on the enzyme conformation state (Redko, Y. et al., Molecular Microbiology, 2009) 71(5), 1145-1154). It has been shown that in the plastids of plant cells Mini-III regulates the amount of introns and non-coding RNA molecules present in the cell by degradation thereof (Hotto A., et al., Plant Cell, (2015), 27(3), 724-740).

The inventors' team, for the first time in history, has described the sequence specific activity of RNase towards double-stranded RNA (dsRNA) exhibited by the Mini-III enzyme and its mutant D94R. The reported results confirm the sequence-dependent cleavage of long dsRNA molecules by Mini-III RNase from Bacillus stubtilis (BsMiniIII). The analysis of sites cleaved by this enzyme during limited cleavage of long dsRNA molecules, bacteriophage φ6 genome, has led to the identification of a sequence motif in dsRNA cleaved by this enzyme. Moreover, nucleotide residues within the cleavage site have been established which affect the cleaving efficiency and are essential for the enzyme to recognize the dsRNA sequence. Structural studies followed by the computer modelling backed up with suitable experiments, have also shown that the α5b-α6 loop, a structural element characteristic for enzymes belonging to Mini-III RNases, plays a key role in specific activation of the protein, but not in the process of binding dsRNA (Glow D., et al., Nucleic Acids Res, (2015), 43(5), 2864-2873).

The identification of other RNases exhibiting sequence specificity towards dsRNA and the possibility to change the specificity thereof would enable development in the field of RNA nucleic manipulation techniques, as well as the development of novel research methods and applications with such enzymes, and new technologies using such enzymes.

Based on the above premises, the aim of the invention is, firstly, to provide dsRNA RNases with high sequence specificity, other than BsMiniIII, recognizing and cleaving a particular sequence in double-stranded RNA, and secondly, in order to change substrate preference and activity thereof, to provide a method based on the exchange of defined structural elements between the enzymes of this endonuclease subfamily. The aim of the invention is also to provide a method of determining, isolating, obtaining, selecting, and preparing such sequence-specific dsRNA RNases.

While testing the activity and specificity of a set of BsMiniIII homologues, the inventors have unexpectedly found that the enzymes of ribonuclease Mini-III family have different a nucleotide preference during dsRNA cleavage than BsMiniIII. As in the case of BsMiniIII, this preference depends only on the dsRNA sequence and is independent from both an occurrence of irregular helix in dsRNA and cooperation with other proteins. Unexpectedly, the inventors have found that the enzymes of the ribonuclease III superfamily, which in in silico modelling have a loop formed by a polypeptide chain fragment, located in and interacting with the major groove of dsRNA helix, show an activity which allows for specific and defined fragmentation of dsRNA, with properties close to these of restriction enzymes for dsDNA. For the first time, the studies conducted by the inventors have provided evidence for the dependence of cleavage preference on the α5b-α6 loop sequence. Furthermore, the model analysis surprisingly allowed us to find an additional structural element—an α4 helix which, similarly to the mentioned loop, is located suffficently close to the dsRNA sequence and affects the activity and specificity of Mini-III enzymes. The exchange of structural elements between the proteins unexpectedly yielded a change in both specificity and activity of resulting chimeric proteins.

### DISCLOSURE OF INVENTION

The object of the invention concerns a group of sequence-specific Mini-III RNases as well as defining structural elements in Mini-III RNases responsible for sequence preference of these enzymes, and a method of exchanging these elements or fragments thereof between Mini-III enzymes.

The invention relates to a Mini-III RNase with amino acid sequence comprising an acceptor part and parts for a transplantable α4 helix and/or a transplantable α5b-α6 loop which form the structures of α4 helix and α5b-α6 loop, respectively, in the Mini-III RNase structure,
wherein the fragments which form the structures of α4 helix and α5b-α6 loop, respectively, correspond structurally to respective structures of α4 helix and α5b-α6 loop formed by amino acid sequence fragments 46-52 and 85-98, respectively, of Mini-III RNase from Bacillus stubtilis shown in SEQ ID NO: 1,
wherein the said Mini-III RNase shows sequence specificity in dsRNA cleavage being dependent only on aribonucleotide sequence of a substrate and independent of an occurrence of secondary structures in the substrate's structure, and independent of a presence of other assisting proteins, and wherein the Mini-III RNase is not the Mini-III protein from Bacillus stubtilis of SEQ ID NO: 1, nor of SEQ ID NO: 1 with D94R mutation.

In a preferred Mini-III RNase, the amino acid sequence is constructed of an acceptor part, derived from a Mini-III RNase of one microorganism, with inserted transplantable α4 helix and/or a transplantable α5b-α6 loop derived from an α4 helix and/or an α5b-α6 loop sequence respectively from a Mini-III RNase from a different microorganism.

In a preferred Mini-III RNase, the amino acid sequence includes
an acceptor part derived from Mini-III RNase of BsMiniIII^{wt} (SEQ ID NO: 1), or Mini-III CkMiniIII^{wt} of Caldicellulosiruptor kristjanssonii (SEQ ID NO: 2), or CrMiniIII^{wt} of Clostridium ramosum (SEQ ID NO: 4), or CtMiniIII^{wt} of Clostridium thermocellum (SEQ ID NO: 6), or FpMiniIII^{wt} of Faecalibacterium prausnitzii (SEQ ID NO: 8), or FnMiniIII^{wt} of Fusobacterium nucleatum subsp. nucleatum (SEQ ID NO: 10), or SeMiniIII^{wt} of Staphylococcus epidermidis (SEQ ID NO: 12), or TmMiniIII^{wt} of Thermotoga maritima (SEQ ID NO: 14), or TtMiniIII^{wt} of Thermoanaerobacter tengcongensis, presently Caldanaerobacter subterraneus subsp. tengcongensis (SEQ ID NO: 16) or an amino acid sequence identical therewith in at least 80%, preferably in 85%, more preferably in 90%, most preferably in 95%;
a transplantable α4 helix derived from BsMiniIII^{wt} with amino acid sequence including amino acids in positions 46-52 of SEQ ID NO: 1, or from CkMiniIII^{wt} with amino acid sequence including amino acids 36-42 of SEQ ID NO: 2, or from CtMiniIII^{wt} with amino acid sequence including amino acids 40-46 of SEQ ID NO: 4, or from CtMiniIII^{wt} with amino acid sequence including amino acids in positions 56-62 of SEQ ID NO: 6, or from FpMiniIII^{wt} with amino acid sequence including amino acids in positions 45-51 of SEQ ID NO: 8, or from FnMiniIII^{wt} with amino acid sequence including amino acids 45-51 of SEQ ID NO: 10, or from SeMiniIII^{wt} with amino acid sequence including amino acids in positions 43-49 of SEQ ID NO: 12, or from TmMiniIII^{wt} with amino acid sequence including amino acids 45-51 of SEQ ID NO: 14, or from TtMiniIII^{wt} with amino acid sequence including amino acids 50-56 of SEQ ID NO: 16) or an amino acid sequence identical therewith in at least 80%, preferably in 85%, more preferably in 90%, most preferably in 95%, and/or
a transplantable α5b-α6 loop derived from BsMiniIII^{wt} with amino acid sequence including amino acids in positions 85-98 of SEQ ID NO: 1, or from CkMiniIII^{wt} with amino acid sequence including amino acids 73-86 of SEQ ID NO: 2, or from CtMiniIII^{wt} with amino acid sequence including amino acids 79-88 of SEQ ID NO: 4, or from CtMiniIII^{wt} with amino acid sequence including amino acids in positions 93-106 of SEQ ID NO: 6, or from FpMiniIII^{wt} with amino acid sequence including amino acids in positions 82-95 of SEQ ID NO: 8, or from FnMiniIII^{wt} with amino acid sequence including amino acids 82-95 of SEQ ID NO: 10, or from SeMiniIII^{wt} with amino acid sequence including amino acids in positions 82-95 of SEQ ID NO: 12, or from TmMiniIII^{wt} with amino acid sequence including amino acids 82-93 of SEQ ID NO: 14, or from TtMiniIII^{wt} with amino acid sequence including amino acids 87-100 of SEQ ID NO: 16, or an amino acid sequence identical therewith in at least 80%, preferably in 85%, more preferably in 90%, most preferably in 95%.

A preferred Mini-III RNase maintains Mini-III RNase activity and includes a sequence or a fragment of an amino acid sequence from Caldicellulosiruptor kristjanssonii shown in SEQ ID NO: 2, wherein the Mini-III RNase shows sequence specificity in dsRNA cleavage and cleaves dsRNA within a consensus sequence

↓
WSSWNNYY
WSSWNNRR
↑

where N=A, C, G, U; W=A, U; S=C, G; Y=C, U.

A preferred RNase maintains Mini-III RNase activity and includes a sequence or a fragment of an amino acid sequence from Clostridium ramosum shown in SEQ ID NO: 4, wherein the Mini-III RNase shows sequence specificity in dsRNA cleavage and cleaves dsRNA within a consensus sequence

```
        ↓
SNWSSW
SNWSSW
    ↑
```

where N=A, C, G, U; W=A, U; S=C, G.

A preferred RNase maintains Mini-III RNase activity and includes a sequence or a fragment of an amino acid sequence from Clostridium thermocellum shown in SEQ ID NO: 6, wherein the Mini-III RNase shows sequence specificity in dsRNA cleavage and cleaves dsRNA within a consensus sequence

↓
WSSW
WSSW
↑

where W=A, U; S=C, G.

A preferred RNase maintains Mini-III RNase activity and includes a sequence or a fragment of an amino acid sequence from Faecalibacterium prausnitzii shown in SEQ ID NO: 8, wherein the Mini-III RNase shows sequence specificity in dsRNA cleavage and cleaves dsRNA within a consensus sequence

↓
WSSW
WSSW
↑

where W=A, U; S=C, G.

A preferred RNase maintains the Mini-III RNase activity and includes a sequence or a fragment of an amino acid sequence from Fusobacterium nucleatum shown in SEQ ID NO: 10, wherein the Mini-III RNase shows sequence specificity in dsRNA cleavage and cleaves dsRNA within a consensus sequence

↓
ASSW
USSW
↑

where W=A, U; S=C, G.

A preferred RNase maintains Mini-III RNase activity and includes a sequence or a fragment of an amino acid sequence from Staphylococcus epidermidis shown in SEQ ID NO: 12, wherein the Mini-III RNase shows sequence specificity in dsRNA cleavage and cleaves dsRNA within a consensus sequence

↓
WNSU
WNSA
↑

where N=A, C, G, U; W=A, U; S=C, G.

A preferred RNase maintains Mini-III RNase activity and includes a sequence or a fragment of an amino acid sequence from Thermotoga maritima shown in SEQ ID NO: 14, wherein the Mini-III RNase shows sequence specificity in dsRNA cleavage and cleaves dsRNA within a consensus sequence

↓
WSSWNG
WSSWNC
↑

where N=A, C, G, U; W=A, U; S=C, G.

A preferred RNase maintains Mini-III RNase activity and includes a sequence or a fragment of an amino acid sequence from Thermoanaerobacter tengcongensis (Caldanaerobacter subterraneus subsp. tengcongensis) shown in SEQ ID NO: 16, wherein the Mini-III RNase shows sequence specificity in dsRNA cleavage and cleaves dsRNA within a consensus sequence

↓
WSSWNNYY
WSSWNNYY
↑

where N=A, C, G, U; W=A, U; S=C, G; Y=C, U.

A preferred RNase is a chimeric protein selected from Ct(FpH) of SEQ ID NO: 18, Ct(FpL) of SEQ ID NO: 20, Ct(FpHL) of SEQ ID NO: 22, Bs(FpH) of SEQ ID NO: 24, Bs(FpL) of SEQ ID NO: 26, Se(FpH) of SEQ ID NO: 28.

The invention also relates to a method of obtaining a Mini-III RNase chimeric protein which includes the steps of:
a) cloning a gene which encodes a Mini-III RNase, wherein the amino acid sequence thereof includes fragments which form structures of α4 helix and α5b-α6 loop, respectively, corresponding structurally to respective fragments in α4 helix and α5b-α6 loop formed by amino acid sequence fragments 46-52 and 85-98, respectively, of Mini-III RNase from Bacillus stubtilis shown in SEQ ID NO: 1,
b) modifying the gene which encodes said RNase by exchanging at least one of the fragments encoding α4 helix and/or α5b-α6 loop structures, respectively, with a fragment encoding α4 helix and/or α5b-α6 loop structures, respectively, from a gene which encodes RNase of a different microorganism,
wherein said Mini-III RNase shows sequence specificity in dsRNA cleavage being dependent only on a ribonucleotide sequence and independent from an occurrence of secondary structures in the substrate's structure, and independent from a presence of other assisting proteins, and wherein the Mini-III RNase is not the Mini-III protein from Bacillus stubtilis with amino acid sequence shown in SEQ ID NO: 1 , nor SEQ ID NO: 1 with D94R mutation.

In a preferred method of obtaining the chimeric Mini-III RNase, step b) relates to an insertion of a transplantable α4 helix and/or a transplantable α5b-α6 loop into the acceptor part, wherein
the acceptor part is derived from Mini-III RNase of BsMiniIII^{wt} (SEQ ID NO: 1), or MiniIII CkMiniIII^{wt} of Caldicellulosiruptor kristjanssonii (SEQ ID NO: 2), or CrMiniIII^{wt} of Clostridium ramosum (SEQ ID NO: 4), or CtMiniIII^{wt} of Clostridium thermocellum (SEQ ID NO: 6), or FpMiniIII^{wt} of Faecalibacterium prausnitzii (SEQ ID NO: 8), or FnMiniIII^{wt} of Fusobacterium nucleatum subsp. nucleatum (SEQ ID NO: 10), or SeMiniIII^{wt} of Staphylococcus epidermidis (SEQ ID NO: 12), or TmMiniIII^{wt} of Thermotoga maritima (SEQ ID NO: 14), or TtMiniIII^{wt} of Thermoanaerobacter tengcongensis, presently Caldanaerobacter subterraneus subsp. tengcongensis (SEQ ID NO: 16) or includes an amino acid sequence identical therewith in at least 80%, preferably in 85%, more preferably in 90%, most preferably in 95%;
the transplantable α4 helix is derived from
BsMiniIII^{wt} with amino acid sequence including amino acids in positions 46-52 of SEQ ID NO: 1, or from CkMiniIII^{wt} with amino acid sequence including amino acids 36-42 of SEQ ID NO: 2, or from CtMiniIII^{wt} with amino acid sequence including amino acids 40-46 of SEQ ID NO: 4, or from CtMiniIII^{wt} with amino acid sequence including amino acids in positions 56-62 of SEQ ID NO: 6, or from FpMiniIII^{wt} with amino acid sequence including amino acids in positions 45-51 of SEQ ID NO: 8, or from FnMiniIII^{wt} with amino acid sequence including amino acids 45-51 of SEQ ID NO: 10, or from SeMiniIII^{wt} with amino acid sequence including amino acids in positions 43-49 of SEQ ID NO: 12, or from TmMiniIII^{wt} with amino acid sequence including amino acids 45-51 of SEQ ID NO: 14, or from TtMiniIII^{wt} with amino acid sequence including amino acids 50-56 of SEQ ID NO: 16) or includes an amino acid sequence identical therewith in at least 80%, preferably in 85%, more preferably in 90%, most preferably in 95%, and/or
the transplantable α5b-α6 loop is derived from
BsMiniIII^{wt} with amino acid sequence including amino acids in positions 85-98 of SEQ ID NO: 1, or from CkMiniIII^{wt} with amino acid sequence including amino acids 73-86 of SEQ ID NO: 2, or from CtMiniIII^{wt} with amino acid sequence including amino acids 79-88 of SEQ ID NO: 4, or from CtMiniIII^{wt} with amino acid sequence including amino acids in positions 93-106 of SEQ ID NO: 6, or from FpMiniIII^{wt} with amino acid sequence including amino acids in positions 82-95 of SEQ ID NO: 8, or from FnMiniIII^{wt} with amino acid sequence including amino acids 82-95 of SEQ ID NO: 10, or from SeMiniIII^{wt} with amino acid sequence including amino acids in positions 82-95 of SEQ ID NO: 12, or from TmMiniIII^{wt} with amino acid sequence including amino acids 82-93 of SEQ ID NO: 14, or from TtMiniIII^{wt} with amino acid sequence including amino acids 87-100 of SEQ ID NO: 16, or includes an amino acid sequence identical therewith in at least 80%, preferably in 85%, more preferably in 90%, most preferably in 95%.

Preferably, in a method of obtaining a chimeric Mini-III RNase the transplantable α4 helix and the transplantable α5b-α6 loop in the gene encoding Mini-III RNase are derived from different microorganisms.

In a preferred method of obtaining a chimeric Mini-III RNase, the gene encoding Mini-III RNase includes any sequence which encodes an amino acid sequence from a group consisting of SEQ ID NO: 18, 20, 22, 24, 26, 28.

A preferred method of obtaining the chimeric Mini-III RNase further includes the steps of c) culturing cells which express the gene from step b), and d) isolating and purifying the expressed protein from step c), and optionally step of e) determining sequence specificity of the protein obtained in step d).

The invention also includes a Mini-III RNase obtained with the method according to the invention.

The invention also relates to a construct which encodes Mini-III RNase, obtained according to the method of the invention.

The invention also relates to a cell which comprises the gene encoding Mini-III RNases according to the invention, or the construct according to the invention.

The invention also relates to a use of Mini-III RNase according to the invention to cleave dsRNA in a manner dependent only on a ribonucleotide sequence and independent of an occurrence of secondary structures in the substrate's structure, and independent of a presence of other assisting proteins.

The invention also relates to a method of cleaving dsRNA in a manner dependent only on a ribonucleotide sequence and independent of an occurrence of secondary structures within substrate structure and independent of a presence of other assisting proteins, wherein the method includes interaction between a dsRNA substrate and the Mini-III RNase according to the invention.

In a preferred method of cleaving dsRNA, the Mini-III RNase includes a sequence from Caldicellulosiruptor kristjanssonii shown in SEQ ID NO: 2, wherein the Mini-III RNase shows sequence specificity in dsRNA cleavage and cleaves dsRNA within a consensus sequence

↓
WSSWNNYY
WSSWNNRR
↑

where N=A, C, G, U; W=A, U; S=C, G; Y=C, U.

In a preferred method of cleaving dsRNA, the Mini-III RNase includes a sequence from Clostridium ramosum shown in SEQ ID NO: 4, wherein the Mini-III RNase shows sequence specificity in dsRNA cleavage and cleaves dsRNA within a consensus sequence

```
        ↓
SNWSSW
SNWSSW
    ↑
```

where N=A, C, G, U; W=A, U; S=C, G.

In a preferred method of cleaving dsRNA, the Mini-III RNase includes a sequence from Clostridium thermocellum shown in SEQ ID NO: 6, wherein the Mini-III RNase shows sequence specificity in dsRNA cleavage and cleaves dsRNA within a consensus sequence

↓
WSSW
WSSW
↑

where W=A, U; S=C, G.

In a preferred method of cleaving dsRNA, the Mini-III RNase includes a sequence from Faecalibacterium prausnitzii shown in SEQ ID NO: 8, wherein the Mini-III RNase shows sequence specificity in dsRNA cleavage and cleaves dsRNA within a consensus sequence

↓
WSSW
WSSW
↑

where W=A, U; S=C, G.

In a preferred method of cleaving dsRNA, the Mini-III RNase includes a sequence from Fusobacterium nucleatum shown in SEQ ID NO: 10, wherein the Mini-III RNase shows sequence specificity in dsRNA cleavage and cleaves dsRNA within a consensus sequence

↓
ASSW
USSW
↑

where W=A, U; S=C, G.

In a preferred method of cleaving dsRNA, the Mini-III RNase includes a sequence from Staphylococcus epidermidis shown in SEQ ID NO: 12, wherein the Mini-III RNase shows sequence specificity in dsRNA cleavage and cleaves dsRNA within a consensus sequence

↓
WNSU
WNSA
↑

where N=A, C, G, U; W=A, U; S=C, G.

In a preferred method of cleaving dsRNA, the Mini-III RNase includes a sequence from Thermotoga maritima shown in SEQ ID NO: 14, wherein the Mini-III RNase shows sequence specificity in dsRNA cleavage and cleaves dsRNA within a consensus sequence

↓
WSSWNG
WSSWNC
↑

where N=A, C, G, U; W=A, U; S=C, G.

In a preferred method of cleaving dsRNA, the Mini-III RNase includes a sequence from Thermoanaerobacter tengcongensis (Caldanaerobacter subterraneus subsp. tengcongensis) shown in SEQ ID NO: 16, wherein the Mini-III RNase shows sequence specificity in dsRNA cleavage and cleaves dsRNA within a consensus sequence

↓
WSSWNNYY
WSSWNNYY
↑

where N=A, C, G, U; W=A, U; S=C, G; Y=C, U.

The invention also relates to a method of obtaining Mini-III RNase, wherein the method includes the steps of:

a) cloning a gene which encodes a Mini-III RNase, wherein amino acid sequence thereof includes fragments that form structures of α4 helix and α5b-α6 loop, respectively, corresponding structurally to structures of α4 helix and α5b-α6 loop formed by amino acid sequence fragments 46-52 and 85-98, respectively, of endoribonuclease Mini-III RNase from Bacillus stubtilis shown in SEQ ID NO: 1, wherein the Mini-III RNase is not the Mini-III protein from Bacillus stubtilis with amino acid sequence shown in SEQ ID NO: 1, nor SEQ ID NO: 1 with D94R mutation,

b) culturing cells which express the gene from step a), and

c) isolating and purifying the expressed protein from step b);

wherein the said Mini-III RNase shows sequence specificity in dsRNA cleavage being dependent only on a ribonucleotide sequence and independent from an occurrence of secondary structures in the substrate's structure, and independent from a presence of other assisting proteins.

In an embodiment of the method of the invention, the gene encoding Mini-III RNase comprises any sequence which encodes an amino acid sequence from a group consisting of SEQ ID NO: 2, 4, 6, 8, 10, 12, 14, 16.

The inventors have determined the structural elements of Mini-III RNase which are responsible for the sequence preference of the enzymes, and these are the α4 helix and α5b-α6 loop (see FIG. 6). It occurred that although the selected fragments of protein Mini-III amino acid sequences corresponding to α4 helix and α5b-α6 loop are characterized by significant differences, on ends thereof there are similar positions, conserved in the course of evolution, which can be identified by amino acid sequence alignment. For sequence fragments intended for the exchange between analysed enzymes to constitute exact equivalents, the borders thereof were set on positions directly adjacent to conserved positions (see Table 11). For example, for BsMiniIII amino acid sequence (SEQ ID NO: 1), these are amino acids in positions 46-52 for α4 helix, and 85-98 for α5b-α6 loop. For CtMiniIII amino acid sequence (SEQ ID NO: 6), these are amino acids in positions 56-62 for α4 helix, and 93-106 for α5b-α6 loop. For FpMiniIII amino acid sequence (SEQ ID NO: 8), these are amino acids in positions 45-51 for α4 helix, and 82-95 for α5b-α6 loop. For SeMiniIII amino acid sequence (SEQ ID NO: 12), these are amino acids in positions 43-49 for α4 helix, and 82-95 for α5b-α6 loop. Both structural elements, with reference to the amino acid sequence of proteins, are marked in FIG. 7.

The inventors have unexpectedly found that it is possible to exchange these key structural elements and obtain enzymes with changed selectivity and/or with various specificity and characteristics of substrate cleavage.

The inventors have developed a method of exchanging the above-mentioned structural elements between Mini-III enzymes which enables obtaining chimeric proteins with changed sequence preference. The essence of the method relates to the production of chimeric proteins based on Mini-III RNase containing the structures of α4 helix and/or α5b-α6 loop from other protein(s). To produce such chimeric proteins, any suitable method of constructing chimeric proteins known by those skilled in the art may be used. For example, the method may include:
a) synthesis of oligonucleotides corresponding to fragments of a gene or genes of a sequence-specific dsRNA endoribonuclease, encoding particular transplantable structural elements—α4 helix and α5b-α6 loop responsible for sequence preference of the Mini-III RNase being a donor;
b) amplification of plasmids used for overproduction of particular Mini-III RNases with omission of a short sequence fragment which encodes the structure element responsible for sequence specificity thereof, intended for the exchange;
c) combination of the two DNA fragments obtained;
d) expression of Mini-III RNase chimeric proteins from the nucleotide sequence obtained in c).
e) determination of changed sequence specificity of the expressed Mini-III RNase chimeric protein.

Such a preferred method of obtaining a change in selectivity in a derivative and/or variant of Mini-III RNase results in obtaining a derivative and/or variant with changed, increased selectivity towards sequence specificity in dsRNA cleavage, and/or changed sequence specificity, and/or changed and/or increased enzymatic activity.

Therefore, in a specific embodiment of the method of the invention, a gene encoding a chimeric Mini-III RNase is constructed using fragments which encode structures of an α4 helix (further referred to as transplantable α4 helix) and a α5b-α6 loop (further referred to as transplantable α5b-α6 loop), respectively, of which at least one is derived from sequences of different Mini-III RNase encoding genes, of which at least one is inserted (transplanted) into an acceptor part derived from Mini-III RNase of another microorganism. Preferably, the gene encoding Mini-III RNase comprises a fragment encoding an α4 helix structure, derived from any of genes from a group comprising genes which encode a Mini-III RNase polypeptide BsMiniIII^{wt} of Bacillus subtilis(SEQ ID NO: 1), CkMiniIII^{wt} of Caldicellulosiruptor kristjanssonii (SEQ ID NO: 2), CrMiniIII^{wt} of Clostridium ramosum (SEQ ID NO: 4), CtMiniIII^{wt} of Clostridium thermocellum (SEQ ID NO: 6), FpMiniIII^{wt} of Faecalibacterium prausnitzii (SEQ ID NO: 8), FnMiniIII^{wt} of Fusobacterium nucleatum subsp. nucleatum (SEQ ID NO: 10), SeMiniIII^{wt} of Staphylococcus epidermidis (SEQ ID NO: 12), TmMiniIII^{wt} of Thermotoga maritima (SEQ ID NO: 14), TtMiniIII^{wt} of Thermoanaerobacter tengcongensis, presently Caldanaerobacter subterraneus subsp. tengcongensis (SEQ ID NO: 16).

Preferably, the gene encoding Mini-III RNase comprises a fragment which encodes an α4 helix structure from BsMiniIII^{wt} with amino acid sequence comprising amino acids in positions 46-52 of SEQ ID NO: 1, or from CkMiniIII^{wt} with amino acid sequence including amino acids 36-42 of SEQ ID NO: 2, or from CtMiniIII^{wt} with amino acid sequence including amino acids 40-46 of SEQ ID NO: 4, or from CtMiniIII^{wt} with amino acid sequence including amino acids in positions 56-62 of SEQ ID NO: 6, or from FpMiniIII^{wt} with amino acid sequence including amino acids in positions 45-51 of SEQ ID NO: 8, or from FnMiniIII^{wt} with amino acid sequence including amino acids 45-51 of SEQ ID NO: 10, or from SeMiniIII^{wt} with amino acid sequence including amino acids in positions 43-49 of SEQ ID NO: 12, or from TmMiniIII^{wt} with amino acid sequence including amino acids 45-51 of SEQ ID NO: 14, or from TtMiniIII^{wt} with amino acid sequence including amino acids 50-56 of SEQ ID NO: 16.

Preferably, the gene encoding Mini-III RNase comprises a fragment encoding an α5b-α6 loop structure, derived from any of genes from a group comprising genes which encode Mini-III RNases BsMiniIII^{wt} of Bacillus stubtilis (SEQ ID NO: 1), CkMiniIII^{wt} of Caldicellulosiruptor kristjanssonii (SEQ ID NO: 2), CrMiniIII^{wt} of Clostridium ramosum (SEQ ID NO: 4), CtMiniIII^{wt} of Clostridium thermocellum (SEQ ID NO: 6), FpMiniIII^{wt} of Faecalibacterium prausnitzii (SEQ ID NO: 8), FnMiniIII^{wt} of Fusobacterium nucleatum subsp. nucleatum (SEQ ID NO: 10), SeMiniIII^{wt} of Staphylococcus epidermidis (SEQ ID NO: 12), TmMiniIII^{wt} of Thermotoga maritima (SEQ ID NO: 14), TtMiniIII^{wt} of Thermoanaerobacter tengcongensis, presently Caldanaerobacter subterraneus subsp. tengcongensis (SEQ ID NO: 16).

Preferably, the gene encoding Mini-III RNase comprises a fragment which encodes an α5b-α6 loop structure from BsMiniIII^{wt} with amino acid sequence comprising amino acids in positions 85-98 of SEQ ID NO: 1, or from CkMiniIII^{wt} with amino acid sequence including amino acids 73-86 of SEQ ID NO: 2, or from CtMiniIII^{wt} with amino acid sequence including amino acids 79-88 of SEQ ID NO: 4, or from CtMiniIII^{wt} with amino acid sequence including amino acids in positions 93-106 of SEQ ID NO: 6, or from FpMiniIII^{wt} with amino acid sequence including amino acids in positions 82-95 of SEQ ID NO: 8, or from FnMiniIII^{wt} with amino acid sequence including amino acids 82-95 of SEQ ID NO: 10, or from SeMiniIII^{wt} with amino acid sequence including amino acids in positions 82-95 of SEQ ID NO: 12, or from TmMiniIII^{wt} with amino acid sequence including amino acids 82-93 of SEQ ID NO: 14, or from TtMiniIII^{wt} with amino acid sequence including amino acids 87-100 of SEQ ID NO: 16.

Preferably, the gene encoding Mini-III RNase comprises any sequence which encodes an amino acid sequence from a group consisting of SEQ ID NO: 18, 20, 22, 24, 26, 28.

The object of the invention is also a method of preparing a Mini-III RNase variant according to the invention with increased selectivity towards sequence specificity in dsRNA cleavage, and/or changed sequence specificity, and/or increased enzymatic activity, wherein this method includes:

a) modifying a gene which encodes said RNase by exchanging at least one of fragments encoding α4 helix and α5b-α6 loop structures, respectively, with a fragment encoding α4 helix and α5b-α6 loop structures, respectively, from another gene which encodes Mini-III RNase,

b) expressing the protein encoded by the gene modified in step a) in a cell culture,

c) isolating and purifying the expressed protein from step b),

d) determining the sequence specificity of the protein obtained in step c).

In a preferred embodiment, the α4 helix structure and the α5b-α6 loop structure are derived from genes of different bacteria species.

The invention also relates to a Mini-III RNase obtained by the method of obtaining a Mini-III RNase variant according to the invention.

The sequence specificity of dsRNA cleavage is to be understood as the ability of Mini-III RNase to recognize and cleave dsRNA being dependent only on a ribonucleotide sequence thereof, and not on an occurrence of a looping-out in one or both strands of dsRNA and/or interaction with other assisting proteins.

The term “Mini-III RNase” or “RNase Mini-III” means a dsRNA ribonuclease of class 4 in RNas-III family. Proteins which belong to this group are homodimers and are composed only of a catalytic domain (RNase III) (Olmedo, G., et al., (2008).

The term “acceptor part”, according to the present specification means an amino acid sequence of one of Mini-III RNases or a sequence at least in 80%, more preferably in 85%, more preferably in 90%, most preferably in 95%, or in higher percentage identical with an amino acid sequence of an acceptor part of one of Mini-III RNases, wherein it is possible to remove partly or entirely the amino acid sequence of α4 helix and/or α5b-α6 loop, and to insert into this/these site(s) a transplantable α4 helix and/or a transplantable α5b-α6 loop derived from a donor.

The term “Mini-III RNase chimeric protein” or “chimeric Mini-III RNase”, according to the present specification, means a construct comprising an amino acid sequence of one of Mini-III RNases (called an acceptor part), or a sequence at least in 80%, more preferably in 85%, more preferably in 90%, most preferably in 95%, or in higher percentage identical with an amino acid sequence of one of Mini-III RNases, wherein the amino acid sequence of α4 helix and/or α5b-α6 loop has been partly or entirely replaced by an analogous amino acid sequence of a transplantable α4 helix and/or a transplantable α5b-α6 loop derived from a Mini-III RNase different than the acceptor part, called the donor, or by a sequence at least in 80%, more preferably in 85%, more preferably in 90%, most preferably in 95%, or in higher percentage identical with the analogous amino acid sequence of α4 helix and/or α5b-α6 loop derived from a different Mini-III RNase.

The term “identity” as used herein refers to a sequence similarity between two polypeptide chains. When the same amino acid residue occupies a position in both compared sequences, then the respective molecules are identical in this position. The percent identity as used herein refers to a comparison between amino acid sequences, and it is determined by comparing two optimally aligned sequences, wherein the part of the amino acid sequence being compared may include additions (i.e. insertion of amino acid residues) or deletions (i.e. removal of amino acid residues) in comparison with the reference sequence (which does not include any additions or deletions) in order to optimally align the two sequences.

As mentioned above, the present invention is based on an unexpected observation that it is possible to construct enzymes which cleave RNA substrates with various sequence specificity in a manner dependent only on the sequence, by manipulating key structural elements—α4 helix and/or α5b-α6 loop, corresponding structurally to the structures of α4 helix and α5b-α6 loop, respectively, formed by fragments with amino acid residues 46-52 and 85-98, respectively, of amino acid sequence of endoribonuclease Mini-III RNase from Bacillus stubtilis shown in SEQ ID NO: 1. For a person skilled in the art it will be obvious that RNases indicated in the examples are only particular embodiments of the present invention. Thus, the present invention also relates to derivatives and/or variants of Mini-III RNases described herein which comprise an amino acid sequence at least in 80%, more preferably in 85%, more preferably in 90%, most preferably in 95% identical with any one of enzymes described herein. In particular, derivatives and/or variants of Mini-III RNases described herein comprise conservative substitutions of corresponding amino acid residues in the reference sequence.

“Conservative substitutions” in the reference sequence are substitutions including amino acid residues which are physically or functionally similar to the corresponding reference residue, e.g. which have similar size, shape, electric charge, chemical properties, including the ability to form covalent or hydrogen bonds, or the like. Particularly, preferred conservative substitutions are the ones which fulfil the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

Mini-III RNases exhibiting sequence specificity and/or chimeric proteins, and/or derivatives and/or variants of Mini-III RNases exhibiting sequence specificity towards dsRNA according to the invention, and the use thereof enable development of a whole new field of RNA manipulation techniques, as well as development of novel research methods and applications of such enzymes and novel technologies utilizing such enzymes. Mini-III RNases showing sequence specificity, and derivatives and/or variants thereof, for example, will be used in ribonucleotide modifications, in structural studies of RNA in order to understand the structure of RNA molecules and/or modifications thereof, in the generation of RNAi molecules, in particular siRNA, in diagnostics and treatment of plant and animal viral diseases, as well as in applications in nanotechnology based on RNA, so-called ‘RNA tectonics’.

New Mini-III RNases showing sequence specificity, and/or chimeric proteins, and/or derivatives and/or variants of Mini-III RNases showing sequence specificity towards dsRNA according to the invention will be used in novel biotechnological applications. There have been known enzymes which cleave single-stranded RNA in a sequence-dependent manner, yet the activity thereof depends not only on the substrate sequence, but also on its structure, thus they are not very useful in practice. In contrast, new Mini-III RNases showing sequence specificity towards dsRNA, and/or chimeric proteins, and/or derivatives and/or variants of Mini-III RNases showing sequence specificity towards dsRNA according to the invention do not have these disadvantages and can be used as laboratory reagents commonly used for dsRNA cleavage, such as restriction enzymes used in molecular biology for dsDNA cleavage. In addition to the in vitro use of Mini-III RNases showing sequence specificity, and/or chimeric proteins, and/or derivatives and/or variants of Mini-III RNases showing sequence specificity towards dsRNA, there is also a possibility to employ these in medicine, diagnostics and nanotechnology. For example, in structural studies of RNA, direct RNA sequencing by reverse transcription reaction is currently the one most used to identify modifications, or mass spectrometry is used for the same purpose. In both cases the analysis of large RNA molecules (e.g., rRNA or mRNA) is problematic. In these methods, nucleases are used to fragment RNA, the cleavage products being short RNA fragments or ribonucleotides. Such cleavage is unspecific, and the multitude of resulting products makes the interpretation of the obtained results difficult or even impossible. The use of new Mini-III RNases showing sequence specificity, and/or chimeric proteins, and/or derivatives and/or variants of Mini-III RNases according to the invention enables cleavage of such RNA molecules into smaller repeatable fragments in a controlled way. Their molar mass and properties may be determined independently, whereby it is possible to perform analyses of ribonucleotide modifications as well as RNA structural studies what thus far has been impossible or very difficult. Such studies of modifications and structures of RNA molecules will provide information on the potential therapeutic targets, like, for example the mechanisms of bacterial resistance to antibiotics. The use of new Mini-III RNases showing sequence specificity, and/or chimeric proteins, and/or derivatives and/or variants of Mini-III RNases according to the invention enables development of technologies based on RNAi, short interfering dsRNA molecules. Mini-III RNases showing sequence specificity, and/or chimeric proteins, and/or derivatives and/or variants of Mini-III RNases showing sequence specificity towards dsRNA according to the invention will be used in methods and applications using siRNA or shRNA for gene silencing, used in medicine, for example, to treat cancers, metabolic and neurodegenerative diseases. Currently, one of the strategies which result in obtaining short double-stranded RNA fragments is to treat long dsRNA, obtained from a particular DNA fragment, with ribonuclease III from Escherichia coli. This enzyme cleaves dsRNA non-specifically, producing fragments of 18 to 25 base pairs. Thus obtained short siRNA fragments are used in gene silencing. The use of Mini-III RNases showing sequence specificity, and/or chimeric proteins, and/or derivatives and/or variants of Mini-III RNases according to the invention gives completely new and unknown possibilities for the production of specific siRNA, enabling the generation of a defined pool of these dsRNA fragments which would most efficiently silence expression of a particular gene, and also would reduce side effects of silencing other genes.

Mini-III RNases showing sequence specificity, and/or chimeric proteins, and/or derivatives and/or variants of Mini-III RNases showing sequence specificity towards dsRNA according to the invention will be applicable in diagnostics, and also in the treatment of diseases caused by viruses with dsRNA as a genetic information carrier. One example of such viruses is rotaviruses from the Reoviridae family, of which three groups are pathogenic for humans. Currently, to detect and identify these groups, reverse transcription followed by PCR technique is used. The availability of Mini-III RNases showing sequence specificity, and/or chimeric proteins, and/or derivatives and/or variants of Mini-III RNases according to the invention enables the manipulation of dsRNA, significantly accelerating the diagnostics. Currently, the treatment of rotavirus infections is highly ineffective. Mini-III RNases showing sequence specificity, and/or chimeric proteins, and/or derivatives and/or variants of Mini-III RNases showing sequence specificity towards dsRNA according to the invention will be used as means for the treatment of diseases caused by rotaviruses, enabling the specific cleavage of viral genome, and thus preventing further replication thereof.

Mini-III RNases showing sequence specificity, and/or chimeric proteins, and/or derivatives and/or variants of Mini-III RNases showing sequence specificity towards dsRNA according to the invention will be also used in nanotechnology, in particular in ‘RNA tectonics’, and the formation of nanostructures based on RNA with given sequence and structure.

Publications cited in the specification as well as references included therein are hereby included herein in their entirety as references.

### BRIEF DESCRIPTION OF DRAWINGS

For better understanding of the invention, it has been illustrated with embodiments and attached figures wherein:

FIG. 1. Differences in the Mini-III RNase cleavage pattern of bacteriophage φ6 genomic dsRNA. M notes a dsDNA marker (Thermo Scientific, SM0223), Ø denotes control reactions without the addition of any enzyme.

FIG. 2. Sequence motifs preferred by particular Mini-III RNases obtained in result of the analysis of data from high throughput sequencing.

FIG. 3. The cleavage of 5 selected phage dsRNA fragments containing sites identified with high throughput sequencing of Mini-III RNase cleavage products. Black asterisks denote substrates, grey asterisk denote a larger of products obtained as a result of substrate cleavage at the expected site.

FIG. 4. The effect of substitutions within central tetranucleotide ACCU positions introduced to 910S substrate on the cleavage efficiency of selected Mini-III RNases in relation to the initial dsRNA sequence. An asterisk denotes sequences complementary to the tetranucleotide sequence of the particular dsRNA substrate.

FIG. 5. The effect of substitutions of selected positions outside the central tetranucleotide (in fig., positions 6, 7, 8, 9) introduced to the 910S substrate on the cleavage efficiency of selected Mini-III RNases. Fragment of substrate's original sequence that contains the cleavage site for Mini-III RNases has been shown at the top of the figure. Also, the numeration of positions and types of substitutions in particular substrates are provided. At the bottom of the figure, the cleavage efficiency for particular substrates in relation to the initial dsRNA sequence is given.

FIG. 6. The theoretical model of BsMiniIII RNase complex with dsRNA. Circles and arrows indicate preferred dsRNA sequence (ACCU) and structural elements responsible for the substrate preference of Mini-III RNase (A-α4 helix, B-α5b-α6 loop).

FIG. 7. Alignment of amino acid sequences of selected Mini-III RNases. Provided numeration of amino acid residues and secondary structure refer to the sequence and secondary structure of BsMiniIII RNase. Conserved catalytic amino acid residues are marked with grey font (D-position 23, and E-position 106). Fragments of structural elements, α4 helix (H) and α5b-α6 loop (L), exchanged during the chimeric protein formation, are denoted with grey background.

FIG. 8. The effect of exchanging the structural elements responsible for Mini-III RNase sequence preference, α4 helix and α5b-α6 loop, on the enzymatic activity of chimeric proteins (A panel) and the sequence preference thereof (B panel).

### MODES FOR CARRYING OUT THE INVENTION

The following examples are included only to illustrate the invention and to explain particular aspects thereof, and not to limit it, and should not be construed as the entire range thereof which is defined in the appended claims.

In the following examples, unless indicated otherwise, standard materials and methods described in Sambrook J. et al., “Molecular Cloning: A Laboratory manual, 2^{nd} edition. 1989. Cold Spring Harbor, N.Y. Cold Spring Harbor laboratory Press” were used, or procedures were followed in accordance with manufacturers' instructions for specific materials and methods.

In the present specification, unless indicated otherwise, standard abbreviations for amino acids and nucleotides or ribonucleotides are used.

### EXAMPLES

### Example 1

The cloning of Sequences Encoding Patricular Mini-III RNases

Microorganisms were purchased in a freeze-dried form from DSMZ (Leibniz Institute DSMZ-German Collection of Microorganisms and Cell Cultures) (Table 1). Following the suspension of lyophilisates in 500 μl of TE buffer (10 mM Tris-HCl pH 8.0, 1 mM EDTA pH 8.0), the suspensions were extracted with phenol (saturated with 100 mM Tris-HCl, pH 8.5). The aqueous phase was re-extracted with phenol:chloroform (1/1 v/v) mixture, and subsequently nucleic acids were precipitated by the addition of 50 μl of 3 M sodium acetate pH 5.2 and 1 mL of ethanol. Precipitated nucleic acids were centrifuged (12 000 g, 10 min., 4° C.), and the fluid was removed. The pellet was washed with 1 mL of 70% ethanol, and then dried. Obtained DNA was suspended in 20 μl of TE buffer.

TABLE 1
The origin of cloned sequences encoding Mini-III RNases

| | | | | | | Optimal |
|---|---|---|---|---|---|---|
| | | | | | | Growth |
| | | | GenInfo Identifier | SEQ ID | DSMZ | Temperature |
| Enzyme | Organism | Strain | (GI) | NO: | No. | (° C.) |
| CkMiniIII | Caldicellulosiruptor | I77R1B | 311792827 | 3 | 12137 | 70 |
| | kristjanssonii | | | | | |
| CrMiniIII | Clostridium ramosum | 113-I | 167756029 | 5 | 1402 | 37 |
| CtMiniIII | Clostridium thermocellum | | 125974551 | 7 | 1237 | 55 |
| FpMiniIII | Faecalibacterium | A2-165 | 160943938 | 9 | 17677 | 37 |
| | prausnitzii | | | | | |
| FnMiniIII | Fusobacterium | 1612A | 19704899 | 11 | 15643 | 37 |
| | nucleatum subsp. | | | | | |
| | nucleatum | | | | | |
| SeMiniIII | Staphylococcus | PCI | 27467211 | 13 | 1798 | 37 |
| | epidermidis | 1200 | | | | |
| TmMiniIII | Thermotoga maritima | MSB8 | 15644486 | 15 | 3109 | 80 |
| TtMiniII | Thermoanaerobacter | MB4 | 20808680 | 17 | 15242 | 75 |
| | tengcongensis, presently | | | | | |
| | Caldanaerobacter | | | | | |
| | subterraneus subsp. | | | | | |
| | tengcongensis (Tte) | | | | | |

TABLE 2
Primer sequences used for the amplification of
genomic DNA encoding particular Mini-III RNases.

| | | SEQ ID |
|---|---|---|
| Primer | Sequence | NO: |
| Fckminilll | CCTCCATGGTCAGTCCTTTAGTATATG | 30 |
| Rckminilll | CCTCTCGAGTTATTGACAGCTATTCTTGGC | 31 |
| Fcrminilll | GGACCATGGGCCCTGAACTGATTAATGC | 32 |
| Rcrminilll | GGCCTCGAGTTATTTGTTGTTGATGTACTG | 33 |
| Fctminilll | CAGGCATATGGTTTGGGAATTTTTTGAC | 34 |
| Rctminilll | GACCTCGAGTCAATTCTGTGAAACAGCC | 35 |
| Ffpminilll | GGACCATGGACGAAAGCGAAAAAATTG | 36 |
| Rfpminilll | GCGCTCGAGTTATTTCTGATCAGGATCAAAC | 37 |
| Ffnminilll | CCGCATATGGACAATGTAGATTTTTCAAAG | 38 |
| Rfnminilll | GTGCTCGAGTCATCATTCTCCCTTTATAAC | 39 |
| | TATATTTATAATTTTTTTTATTTC | |
| Fseminilll | TAGACATATGGCAGTGGCTAAACATATGAAC | 40 |
| Rseminilll | ATCTCGAGCTACCTTTCATCCACTA | 41 |
| Ftmminilll | GCTTCATATGGAAAAACTCTTCAGATTCG | 42 |
| Rtmminilll | CTTCTCGAGTTATTCCTGAGCGCTTCC | 43 |
| Fttminilll | CGCACATATGGAAAAGGATAAGATGATTCTTG | 44 |
| Rttminilll | GCTCTCGAGTCATTCTTCCGTGTATTCCATAG | 45 |

Sequences encoding Mini-III RNases were amplified from genomic DNA in PCR using Pfu polymerase and primers listed in Table 2.

To obtain recombinant plasmids which would enable inducible overexpression of Mini-III RNases with N-terminal hexahistidine tag, PCR products were cleaved with Ndel and Xhol enzymes and ligated with pET28a vector (Novagen) cleaved with the same enzymes. Ligation was conducted for 1 hour at room temperature with 1 U of phage T4 DNA ligase (Thermo Scientific). Next, 10 μl of reaction mixture was used to transform 100 μl of chemically competent bacteria (Escherichia coli Top10 strain: F-mcrA Δ(mrr-hsdRMS-mcrBC) φ80lacZ ΔM15 ΔlacX74 deoR recA1 araD139 Δ(araA-leu)7697 galU galK rpsL endA1 nupG [Invitrogen]), and the transformants were selected on a solid LB medium supplemented with 50 μg/mL kanamycin. From selected clones, plasmid DNA was isolated using Plasmid Mini kit (A&A Biotechnology), followed by sequencing to verify the correctness of the obtained constructs.

In this way, constructs enabling the efficient inducible overproduction of Mini-III RNases from particular microorganism were obtained.

### Example 2

Expression and Purification of Proteins from Recombinant Plasmids Encoding Wild Type Mini-III RNases.

E. coli strain BL21(DE3) (F-ompT gal dcm Ion hsdSB(rB- mB-) λ(DE3 [lad lacUV5-T7 gene 1 ind1 sam7 nin5]) was transformed with recombinant plasmids carrying Mini-III nuclease genes obtained in Example 1. The transformation was performed as in Example 1. Transformants were selected on LB solid medium supplemented with 50 μg/mL kanamycin and 1% glucose. 25 mL of liquid LB medium with 50 μg/mL kanamycin and 1% glucose was inoculated with selected colonies of extransformants and incubated for 5 hours at the temperature of 37° C. with shaking. Then, 500 mL of liquid ZY (Studier 2005) supplemented with kanamycin to the concentration of 100 μg/mL was inoculated with 25 mL of culture grown in LB medium, and incubated with shaking at the temperature of 37° C. for 24 h. The cultures were centrifuged at 5000 g for 10 min at 4° C., suspended in STE buffer (0.1 M NaCl, 10 mM Tris-HCl pH 8.0, 1 mM EDTA pH 8.0), and again centrifuged. The pellet was suspended in 20 mL of lysis solution (50 mM Tris-HCl pH 8.0, 300 mM NaCl, 10 mM imidazole, 10% glycerol), and then bacterial cells were disintegrated with a single pass through the cell disintegrator (Constant Systems LTD) at overpressure of 1360 atmospheres. Lysates were clarified by centrifugation at 20 000 g at the temperature of 4° C. for 20 min to get rid of insoluble cell debris. Recombinant proteins were purified by affinity chromatography method using the polyhistidine tag present in the polypeptide chain.

The cell lysate obtained from a 5 L culture (10 flasks with 500 mL each) was applied to a 7×1.5 cm column containing 5 mL of Ni-NTA agarose bed (Sigma-Aldrich) which had been equilibrated with four volumes of lysis buffer. The column was washed sequentially with the following buffers: lysis (150 mL), lysis supplemented with 2 M NaCl (50 mL), lysis supplemented with imidazole to the concentration of 20 mM (50 mL). Purified recombinant proteins were eluted with lysis buffer supplemented with imidazole to the concentration of 250 mM, and fractions of 1.5 mL were collected. The flow rate during the purification was 0.9 mL/min, and the temperature was 4° C. Purified protein fractions were mixed with equal volume of glycerol, and then stored at −20 ° C.

Thereby, the highly purified enzyme preparations were obtained while maintaining the activity thereof, and in a buffer enabling convenient longer storage thereof at the temperature of −20° C.

### Example 3

Determination of Optimal Reaction Conditions for In Vitro Cleavage of dsRNA Substrates by Purified Enzymes

In order to determine optimal conditions of the reaction buffer, the limited cleavage of c1)6 phage genome was performed in buffers listed in Table 3.

TABLE 3

| Buffer | Buffer Composition |
|---|---|
| B | 10 mM Tris-HCl pH 7.5, 10 mM MgCl₂, 0.1 mg/mL BSA |
| B1 | 10 mM Tris-HCl pH 7.5, 1 mM MgCl₂, 0.1 mg/mL BSA |
| G | 10 mM Tris-HCl pH 7.5, 10 mM MgCl₂, 50 mM NaCl, 0.1 mg/mL BSA |
| G1 | 10 mM Tris-HCl pH 7.5, 1 mM MgCl₂, 50 mM NaCl, 0.1 mg/mL BSA |
| O | 50 mM Tris-HCl pH 7.5, 10 mM MgCl₂, 100 mM NaCl, 0.1 mg/mL BSA |
| O1 | 50 mM Tris-HCl pH 7.5, 1 mM MgCl₂, 100 mM NaCl, 0.1 mg/mL BSA |
| R | 10 mM Tris-HCl pH 8.5, 10 mM MgCl₂, 100 mM KCl, 0.1 mg/mL BSA |
| R1 | 10 mM Tris-HCl pH 8.5, 1 mM MgCl₂, 100 mM KCl, 0.1 mg/mL BSA |
| Y | 33 mM TRIS acetate pH 7.9, 20 mM Mg(CH₃COO)₂, 66 mM CH₃CO₂K, |
| | 0.1 mg/mL BSA |
| 2XY | 66 mM TRIS acetate pH 7.9, 40 mM Mg(CH₃COO)₂, 132 mM CH₃CO₂K, |
| | 0.2 mg/mL BSA |
| Bs | 10 mM Tris-HCl pH 7.5, 1 mM MgCl₂, 5 mM NaCl, 0.1 mg/mL BSA |
| BG | 10 mM Tris-HCl pH 7.5, 10 mM MgCl₂, 25 mM NaCl, 0.1 mg/mL BSA |

In the experiment, 1.5 μg of dsRNA was used, and 3.3 μg of BsMiniIII, 80 ng of CkMiniIII, 23.5 μg of CrMiniIII, 5 μg of CtMiniIII, 0.8 μg of FnMiniIII, 1.1 μg of FpMiniIII, 2.1 μg of SeMiniIII, 0.185 μg of TmMiniIII, 11.5 ng of TtMiniIII, obtained in Example 2. Aliquots corresponding to 0.5 μg dsRNA were collected after 5, 10, and 15 minutes of reaction, except for the TtMiniIII and SeMiniIII, where reaction times were 2, 4 and 6 minutes, and 20, 40 and 60 minutes, respectively.

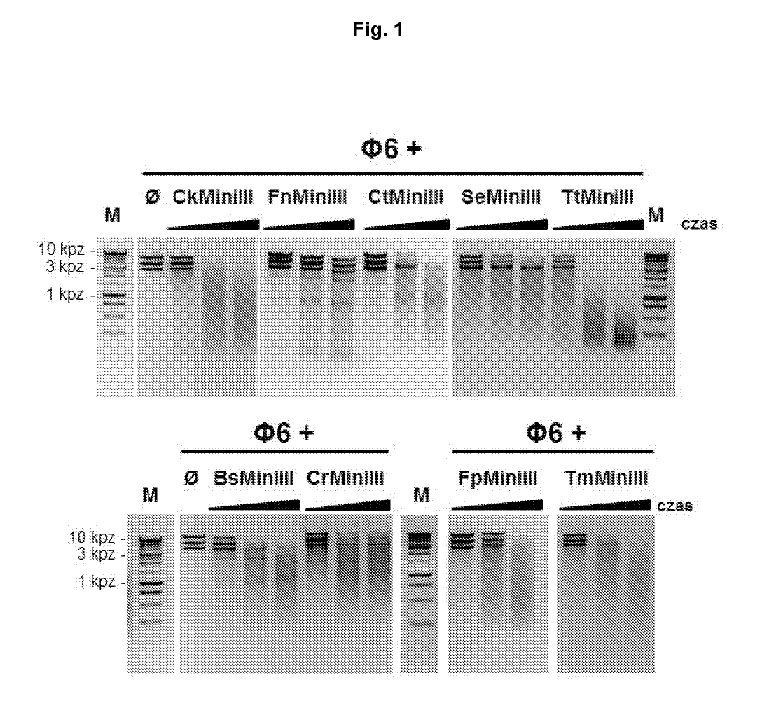

Cleavage products were separated by electrophoresis in 1.5% agarose gel supplemented with a final concentration of 0.5 μg/mL ethidium bromide. The buffer which generated the most visible band pattern was selected as an optimal one. As a result the following buffers were selected: for CrMiniIII—Bs buffer; for CkMiniIII—G1 buffer; for CtMiniIII—B1 buffer; for FpMiniIII—Bs buffer; for FnMiniIII—B buffer; for SpMiniIII—R buffer; for TmMiniIII—R buffer; for TtMiniIII—B1 buffer. In these conditions, clear differences were observed in the pattern of bands obtained in electrophoretic separation of cleavage products (FIG. 1), which reflects differences in the sequence preference of the analysed enzymes.

In this way, convenient conditions for using purified enzymes in in vitro reactions were determined, and the presence of differences in sequence specificities thereof between the particular Mini-III RNases was shown.

### Example 4

Determination of a Preferred Cleavage Sequence for Particular Mini-III RNases using high throughput Sequencing

Limited cleavage of 5 μg of φ6 genome was performed with each enzyme for 5 min. (conditions are given in Table 4). The amount of particular enzymes used in reactions were as given in Example 3.

TABLE 4
Optimal reaction conditions for particular sequence
specific Mini-III RNases.

| | Buffer | Reaction |
|---|---|---|
| | (composition given | Temperature |
| Enzyme | in Table 3 above) | [° C.] |
| BsMiniIII | Bs | 37 |
| CrMiniIII | Bs | 37 |
| CkMiniIII | G1 | 65 |
| CtMiniIII | B1 | 55 |
| FpMiniIII | Bs | 37 |
| FnMiniIII | F | 37 |
| SeMiniIII | R | 37 |
| TmMiniIII | R | 65 |
| TtMiniIII | Bs | 65 |

The reactions were quenched by the addition of EDTA to the concentration of 20 mM, and subsequently RNA was purified using GeneJET RNA Cleanup and Concentration Micro Kit (Thermo Scientific). Purified reaction products were subjected to denaturation at 95° C. for 1 min, cooled on ice, and next 3′ RNA ends were ligated with 50 pmols of 5′ pre-adenyiated adapters UniShPreA (Table 5) using truncated RNA K2270 ligase II (New England Biolabs) at the temperature of 16° C. for 16 hours. Ligation products were purified from unused adapters using GeneJET RNA Cleanup and Concentration Micro Kit (Thermo Scientific), and next they were used as a template in reverse transcription reactions using Maxima reverse transcriptase (Thermo Scientific) and UniShRT primer complementary to UniShPreA sequence (Table 5). Reactions were incubated for 5 minutes at the temperature of 50° C. and terminated by heating for 5 minutes at the temperature of 80° C. The obtained cDNA was purified using GeneJET DNA Micro Kit (Thermo Scientific), and then 3′-ends were ligated with 50 pmols of pre-adenylated adapters PreA3Univ (Table 5) employing thermostable ligase App DNA/RNA (New England Biolabs). Ligation products were purified with GeneJET DNA Micro Kit. Thus prepared double-stranded cDNA was amplified with PCR (15-18 amplification cycles) using a pair of primers/adapters (Table 5) which enabled the sequencing of obtained products in MiSeq sequencer (Illumine). PCR products were separated on 1.5% agarose gel, and a fraction of size between 200 and 700 base pairs was re-isolated using GeneJET Gel Extraction kit (Thermo Scientific).

TABLE 5
Primer sequences used to prepare libraries for
high throughput sequencing of dsRNA ends
resulting from the cleavage of φ6 bacteriophage
genome by sequence-specific Mini-III RNases.

| | | SEQ |
|---|---|---|
| | | ID |
| Primer | Sequence | NO: |
| UniShPreA | AGATCGGAAGAGCGTCGTGTAGGGAAAGAGTGTAGA | 46 |
| UniShRT | TCTACACTCTTTCCCTACACGAC | 47 |
| PreA3Univ | GATCGGAAGAGCACACGTCTGAACTCCAGTCAC | 48 |

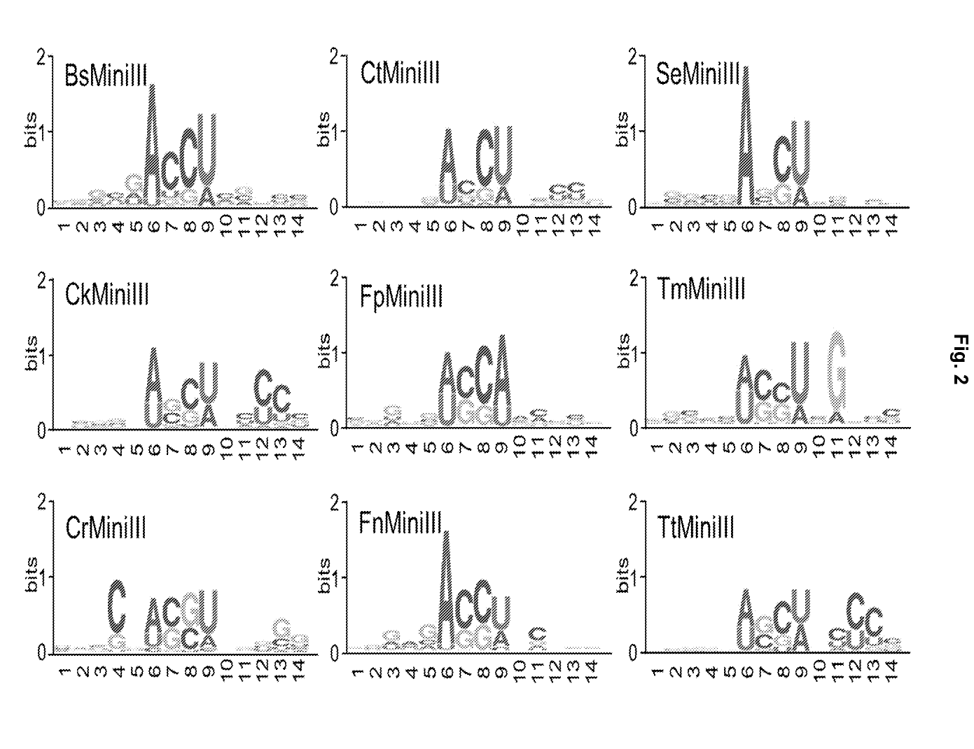

The prepared material was subjected to the high throughput sequencing using MiSeq sequencer (Illumine). Reads obtained from this analysis were aligned to c1)6 bacteriophage genome sequence with Bowtie 2 software (version 0.2), available on the Galaxy platform (http://usegalaxy.ora), using end-to-end mode and default parameters. Taking into account the geometry of substrate cleavage by Mini-III, the total number of reads starting in position X of “+” strand and position X+1 of “−” strand was calculated. In this way, we established a rating of cleaving particular sites in 06 bacteriophage aenome for each enzyme. To determine the substrate preference of each enzyme, we used 14-nucleotide sequence fragments flanking 200 most frequent cleavage sites. In this analysis a software for finding de nova motifs—MEME2 was used with parameters set to default except for the minimum width of motifs which was set for 14 nucleotides. The profiles established for the preferred cleavage sequence for the tested Mini-III RNases are shown in FIG. 2. In this way, the sequence preferences of the tested Mini-III RNases have been characterised.

### Example 5

Cleavage of Isolated dsRNA Substrates Produced Using RT PCR and Enzymatic Synthesis with Mini-III RNases

To confirm the results obtained from the analysis of data from high throughput sequencing of products of RNA cleavage by Mini-III nucleases, short fragments of bacteriophage genome were synthesized comprising potential cleavage sites for Mini-III nucleases (Table 6). To achieve this, reverse transcription of φ6 dsRNA genome was performed using Maxima reverse transcriptase (Thermo Scientific) and random six-nucleotide primers. Subsequently, based on the high throughput sequencing results, sites in bacteriophage genome were selected and pairs of primers were designed to enable amplification of fragments comprising these sites in dsRNA. One of the primers introduced the promoter sequence for phage T7 DNA-dependent RNA polymerase, while the other—the promoter sequence for phage c1)6 RNA-dependent RNA polymerase (Table 7).

TABLE 6
dsRNA substrates - isolated short fragments of bacteriophage
Φ6 genome comprising potential cleavage sites for Mini-III
nucleases (given nucleotide positions are in reference to Φ6
genome sequence) (references to Φ6 genome sequence: S:
NC_003714.1; L: NC_003714; M: NC_003714.3; McGraw,
T. et al., Journal of Virology, (1986) 58(1), 142-151;
Gottlieb, P. et al., Virology, (1988) 163(1), 183-190;
Mindich, L. et al., Journal of Virology, (1988) 62(4),
1180-1185, respectively).

| | | Fragment | Fragment |
|---|---|---|---|
| | Φ6 | Starting | Ending |
| Cleavage | Genome | Nucleotide | Nucleotide |
| Position | Fragment | Position | Position |
| 910 | S | 804 | 948 |
| 949 | S | 910 | 998 |
| 2021 | L | 1786 | 2112 |
| 3292 | L | 3041 | 3384 |
| 4486 | L | 4421 | 4569 |
| 4754 | L | 4650 | 4928 |

TABLE 7
Sequences of primers used to prepare dsRNA being isolated
fragments of φ6 bacteriophage genome. For specific
reactions, pairs of primers marked with substrate cleavage
position were used.

| Cleavage | | | SEQ ID |
|---|---|---|---|
| Position | Primer | Sequence | NO: |
| 910 | 910fT7 | TAATACGACTCACTATAGGGCTGCTCGCGCGTTG | 49 |
| | 910rP6 | GGAAAAAAATCAGACACAACTGACGCGATCG | 50 |
| 949 | 949fT7 | TAATACGACTCACTATAGGGCCTCTCTCTCTGGCCACGATC | 51 |
| | 949rP6 | GGAAAAAAATGCCCTGTACAGCAGGCATAAG | 52 |
| 2021 | 2021fT7 | TAATACGACTCACTATAGGGCTCCTATCATGGCCGTTGC | 53 |
| | 2021rP6 | GGAAAAAAACTTCGAGATCAGGGTTGGACG | 54 |
| 3292 | 3292fT7 | TAATACGACTCACTATAGGGTACCGCGATCAACACTGTCGTC | 55 |
| | 3292rP6 | GGAAAAAAACGAATCAGGACGTCTGGACG | 56 |
| 4486 | 4486fT7 | TAATACGACTCACTATAGGGCTGTCTCCCCTCGGTTTCATC | 57 |
| | 4486rP6 | GGAAAAAAATCGACAGACGACAGCGCTG | 58 |
| 4754 | 4754fT7 | TAATACGACTCACTATAGGGCTCATCGCCTCGATGAACCAAG | 59 |
| | 4754rP6 | GGAAAAAAACTACTGCTTTCGAGCGGTCG | 60 |

dsRNA synthesis reaction was performed using Replicator RNAi Kit (Thermo Scientific) according to the protocol recommended by the manufacturer. The concentration of products of the synthesis reaction was measured spectrophotometrically.

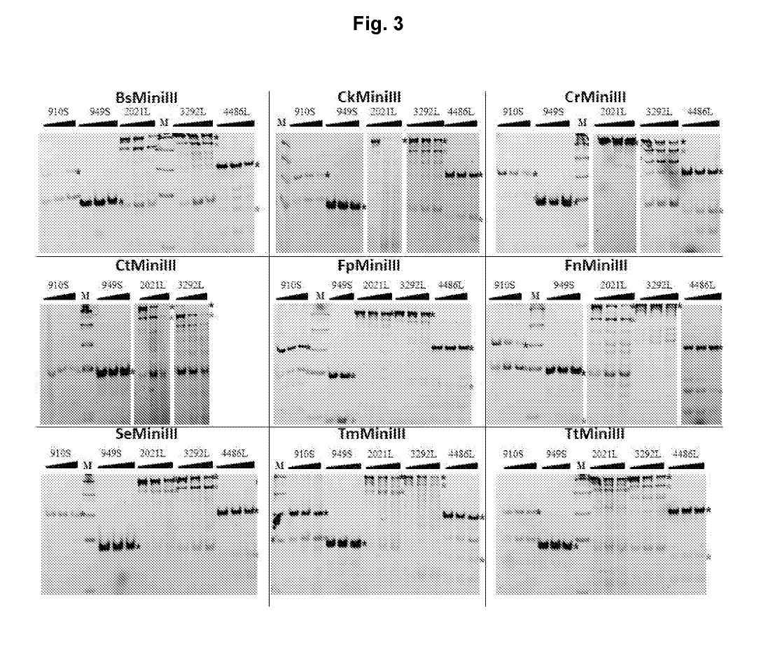

To cleave the substrates being isolated φ6 bacteriophage genome fragments, the panel of described enzymes was used in their optimal conditions described in Table 4. Cleavage products were separated by electrophoresis using polyacrylarnide gel (8%, TAE: 40 mM Tris-HCl, 20 mM acetic acid, and 1 mM EDTA), stained with ethidium bromide for 10 minutes, and visualized using UV light, The results of cleavage of selected substrates are shown in FIG. 3.

In this way, the results of high throughput sequencing and applicability of described method to identify cleavage sites for Mini-III RNases in cl=.6 bacteriophage genome were confirmed.

### Example 6

Preparation of Substrate Libraries with Substitutions and Generation of dsRNA Substrates with Introduced Substitutions

To generate template DNA for dsRNA 910S synthesis, and to introduce a substitutions of a motif recognised by Mini-III RNases, dsDNA obtained in RT-PCR of bacteriophage genorne fragment comprising a fragment of phage genome S segment from position 804 to position 948 was inserted to Sinal site in pUC19 plasmid, whereby pUC910S plasmid was generated. Substitutions at each position of the cleavage site were obtained using inside-out PCR, in which pUC910S plasmid was used as a template, as well as three sets of primers comprising degenerate sequence at the positions subjected to changes (Table 8). Substitutions in positions outside the cleavage site were obtained using inside-out PCR, in which pUC910S plasmid was used as a template, as well as a pair of primers introducing a single substitution outside ACCU sequence (Table 8).

TABLE 8
Sequences of primers used for the construction of
substrate libraries with substitutions and
production of the dsRNA substrates with introduced
substitutions. To generate suitable libraries,
pairs of primers were used, of which one has
f and the other r at the end of their names.
Symbols N, W, S mean as follows: N = A, C, G, U;
W = A, T; S = C, G.

| | | | SEQ |
|---|---|---|---|
| | | | ID |
| Library | Primer | Sequence | NO: |
| 1 | WSSWf | SSWCTCTCTCTGGCCACGATC | 61 |
| | WSSWr | WTTCCCTCCCAGCACG | 62 |
| 2 | ANNTf | NNTCTCTCTCTGGCCACGATC | 63 |
| | ANNTr | TTTCCCTCCCAGCACG | 64 |
| 3 | NCCNf | CCNCTCTCTCTGGCCACGATC | 65 |
| | NCCNr | NTTCCCTCCCAGCACG | 66 |
| 4 | 3T-r | TTTACCTCCCAGCACGACCGCGAC | 67 |
| | 3T-f | CCTCTCTCTCTGGCCACGATCGCGTC | 68 |
| 5 | 4C-r | GCCCTCCCAGCACGACCGCGA | 69 |
| | 4G-r | CCCCTCCCAGCACGACCGCGAC | 70 |
| | 4T-r | ACCCTCCCAGCACGACCGC | 71 |
| | 4-f | AACCTCTCTCTCTGGCCACGATCGCGTC | 72 |
| 6 | 11C-r | CCTCCCTCTCTGGCCACGATCGCGTCAG | 73 |
| | 11G-r | CCTCGCTCTCTGGCCACGATCGCGTCAG | 74 |
| 7 | 12A-r | CCTCTATCTCTGGCCACGATCGCGTCAG | 75 |
| | 11/12-f | TTTCCCTCCCAGCACGACCGCGAC | 76 |

5′-ends of obtained PCR products were phosphorylated using T4 polynucleotide kinase (Thermo Scientific), and circular molecules were reconstructed using phage T4 DNA Haase (Thermo Scientific). Obtained plasmids were subjected to DNA sequencing in order to characterise substitution(s) in particular clones. Selected clones (Tables 9 and 10) comprising a change in motif sequence recognised by Mini-III were used for dsRNA synthesis using Replicator RNAi Kit (Thermo Scientific).

TABLE 9
Nucleotide sequences at the preferred site in
dsRNA substrates from the panel of substrates
comprising substitutions in the sequence
of recognised motif.

| Substrate | Sequence | Complementary Sequence |
|---|---|---|
| S910ACCU | ACCU | AGGU |
| S910AGGU | AGGU | ACCU |
| S910GCCU | GCCU | AGGC |
| S910ACCG | ACCG | CGGU |
| S910AGGA | AGGA | UCCU |
| S910UGGU | UGGU | ACCA |
| S910ACGU | ACGU | ACGU |
| S910ACUU | ACUU | AAGU |
| S910AGAU | AGAU | AUCU |
| S910UCCA | UCCA | UGGA |
| S910UGGA | UGGA | UCCA |
| S910CCCA | CCCA | UGGG |
| S910UCCG | UCCG | CGGA |
| S910GCCG | GCCG | CGGC |
| S910CCCG | CCCG | CGGG |
| S910UCGU | UCGU | ACGA |
| S910AAAU | AAAU | AUUU |
| S910AGUU | AGUU | AACU |
| S910UCGA | UCGA | UCGA |
| S910UGCA | UGCA | UGCA |

TABLE 10
Nucleotide sequences at the preferred site in
dsRNA substrates from clones comprising
substitutions outside ACCU sequence in the
recognised motif. ACCU sequences and
complementary sequence AGGU are in bold.

| Substrate | Sequence | Complementary sequence |
|---|---|---|
| 910-3G-U | UAAACCUCUC | GAGAGGUUUA |
| 910-4A-C | GCAACCUCUC | GAGAGGUUGC |
| 910-4A-G | GGAACCUCUC | GAGAGGUUCC |
| 910-4A-U | GUAACCUCUC | GAGAGGUUAC |
| 910-11U-A | GAAACCUCAC | GTGAGGUUUC |
| 910-11U-G | GAAACCUCGC | GCGAGGUUUC |
| 910-12C-A | GAAACCUCUA | TAGAGGUUUC |

In this way, a panel of substrates was obtained enabling precise and systematic determination of the sequence preferences of Mini-III RNases in a strictly controlled system.

### Example 7

### The Effect of Substitutions in a Recognised Sequence on Cleavage Rate of Selected Mini-III RNases

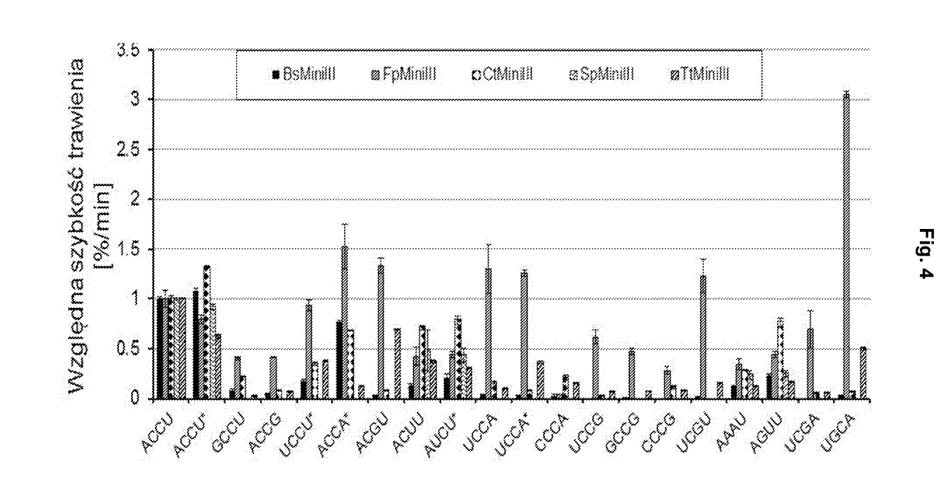

The synthesized panel of 910S substrates comprising substitutions in ACCU sequence, shown in Table 9, was used to investigate the cleavage rate of selected Mini-III enzymes. The reactions were performed in conditions optimal for a particular enzyme described in a table (Table 4). To each reaction, 1.2 μg of dsRNA was added. Subsequently, after 15, 30, 60, and 120 minutes from the reaction initiation, 15 μl aliquots were collected and mixed with 3 μl of loading dye and 1.5 μl of phenol:chloroform (1:1 v:v) mixture, followed by cooling the sample on ice. Polyacrylamide gel (8%, TAE: 40 mM Tris-HCl, 20 mM acetic acid, and 1 mM EDTA) was loaded with 5μl of thus obtained mixture, followed by electrophoresis, gel staining with ethidium bromide (0.5 μg/ml) for 10 minutes, and visualisation of RNA using UV light. The molar ratio of product to substrate was determined densitometrically by measuring the intensity of a band corresponding to the substrate and to the larger of reaction products using ImageQuantTL software (GE Healtcare). The rate was determined from a range for which the reaction was linear in time. Next, the obtained values were normalised to the initial rate of cleaving a substrate comprising ACCU sequence. The results are shown in FIG. 4.

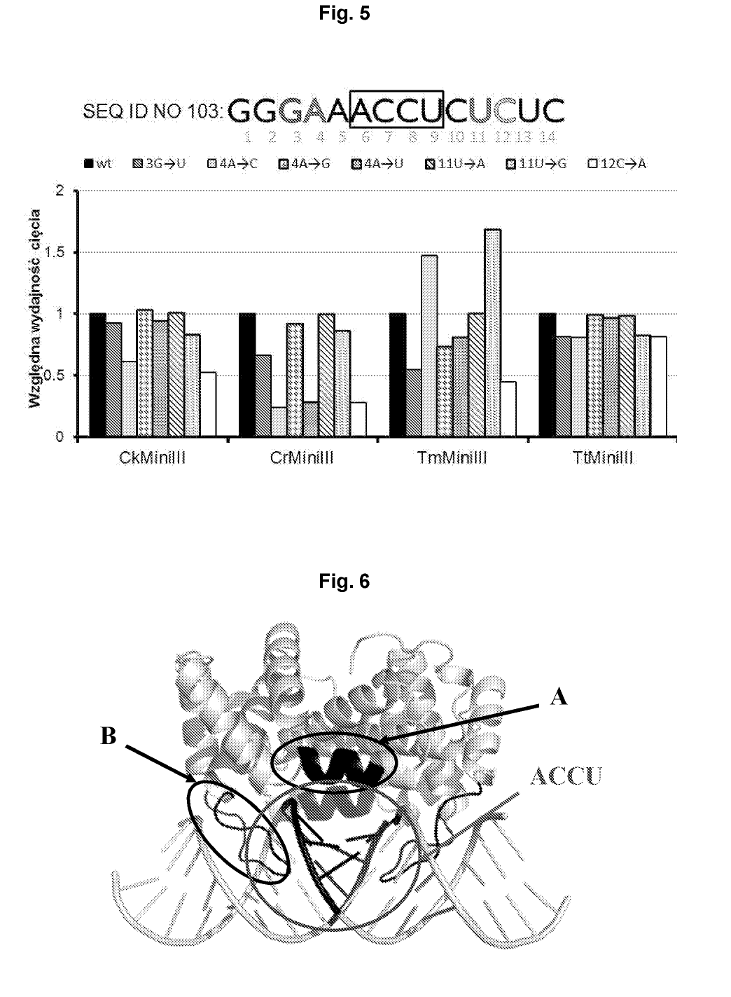

The synthesized panel of 910S substrate variants comprising substitutions outside ACCU sequence, shown in Table 10, was used to investigate the cleavage efficiency of selected Mini-III enzymes. Selection of enzymes was performed based on the analysis of high throughput sequencing results. In this experiment, we used enzymes with recognised sequence motif containing, in addition to four main nucleotides, also nucleotides outside of this sequence. Reactions proceeded as in the case of 910S substrates comprising substitutions in ACCU sequence, wherein reactions were terminated after 60 minutes from the initiation thereof. The cleavage efficiency was determined by dividing the percentage amount of the larger of products by the number of minutes of reaction. Next, the obtained values were normalised to the initial rate of cleaving a substrate comprising ACCU sequence. The results are shown in FIG. 5.

In this way, precise sequence preferences of tested enzymes were established.

### Example 8

The Use of the Model of dsRNA-BsMiniIII Complex Structure for the Selection of Structural Elements Which may be Engaged in Recognising the Preferred Sequence

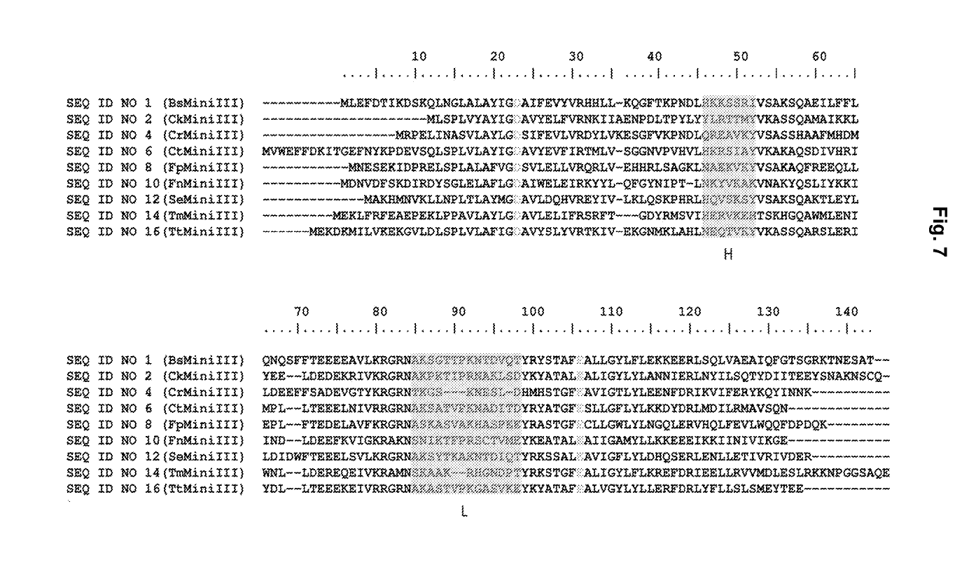

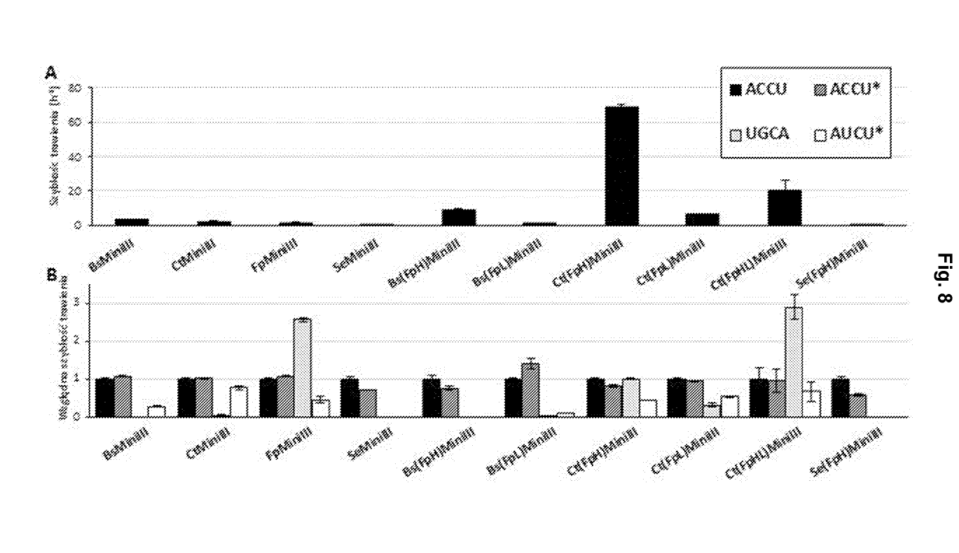

As a result of the visual analysis of the model of BsMiniIII with dsRNA dimer complex structure, it was found that two elements of the structure are located close enough to the RNA substrate to participate in the selection of the substrate sequence to cleave, and the differences in the amino acid sequence of these two elements can be responsible for the differences observed among the tested enzymes in preference towards various substrates. Selected structure elements are α4 helix and α5b-α6 loop (FIG. 6). One of the methods for experimental verification of roles played by these structural elements is to test the effect of a change in the amino acid sequence of these Mini-III regions on the preference to cleave different sequences. To establish precisely the functional region, an alignment of amino acid sequences was done for the enzymes described in Example 1. It occurred that although the selected fragments of protein Mini-III amino acid sequences corresponding to α4 helix and α5b-α6 loop are characterized by significant differences on the sequence level, on ends thereof there are similar positions, conserved in the course of evolution. For sequence fragments, intended for the exchange between analysed enzymes, to constitute exact equivalents, the borders thereof were set on positions directly adjacent to conserved positions. For BsMiniIII amino acid sequence (SEQ ID NO: 1), these are amino acids 46-52 for α4 helix, and 85-98 for α5b-α6 loop. For CtMiniIII amino acid sequence (SEQ ID NO: 6), these are 56-62 for α4 helix, and 93-106 for α5b-α6 loop. For FpMiniIII amino acid sequence (SEQ ID NO: 8), these are 45-51 for α4 helix, and 82-95 for α5b-α6 loop. For SeMiniIII amino acid sequence (SEQ ID NO: 12), these are 43-49 for α4 helix, and 82-95 for α5b-α6 loop. Both structural elements, with reference to the amino acid sequence of proteins, are marked in FIG. 7. Positions of flanking amino acids are shown in Table 11.

TABLE 11
Equivalent amino acid flanking sequences of α4 helix and α5b-α6 loop in
particular Mini-III which may be engaged in recognising the preferred sequence.

| | | Sequences of α4 helix | Sequences of α5b-α6 loop |
|---|---|---|---|
| | | fragments participating in | fragments participating in |
| | | sequence recognition (the | sequence recognition (the range |
| | | range of given positions for | of given positions for amino acid |
| | | amino acid residues is in | residues is in reference to the |
| Enzyme | SEQ ID NO | reference to the SEQ ID NO) | SEQ ID NO) |
| BsMiniIII | 1 | HKKSSRI (46-52) | AKSGTTPKNTDVQT (85-98) |
| CkMiniIII | 2 | YLRTTMY (36-42) | AKPKTIPRNAKLSD (73-86) |
| CrMiniIII | 4 | QREAVKY (40-46) | TKGSKNESLD (79-88) |
| CtMiniIII | 6 | HKRSIAY (56-62) | AKSATVPKNADITD (93-106) |
| FpMiniIII | 8 | NAEKVKY (45-51) | ASKASVAKHASPEE (82-95) |
| FnMiniIII | 10 | NKYVKAK (45-51) | SNIKTFPRSCTVME (82-95) |
| SeMiniIII | 12 | HQVSKSY (43-49) | AKSYTKAKNTDIQT (82-95) |
| TmMiniIII | 14 | HERVKEH (45-51) | SKAAKRHGNDPT (82-93) |
| TtMiniIII | 16 | NEQTVKY (50-56) | AKASTVPKGASVKE (87-100) |

In this way, amino acid positions were selected, which define an optimal sequence region for the exchange of elements responsible for sequence specificity between enzymes.

### Example 9

### The Method for Exchanging Structural Elements Between Wild Type Mini-III RNases

Recombinant plasmids used for overproduction of particular enzymes (FpMiniIII, CtMiniIII, BsMiniIII, and SpMiniIII) were amplified in PCR so as to obtain a product comprising the whole plasmid used as a template with the omission of a short fragment of the sequence encoding the structure element to be exchanged (transplanted). Sequences of used primers are given in Table 12.

TABLE 12
Sequences of primers used for the construction of particular
chimeric proteins. Each pair of primers shown in the table
was used in separate PCR reaction.

| Primer | | | |
|---|---|---|---|
| Pair | Primer | Sequence | SEQ ID NO: |
| CtR1 | CtR1Up | GCTTCATAAGCGCTCCATTGCT | 77 |
| | CtR1Dw | AGCAATGGAGCGCTTATGAAGC | 78 |
| CtR2 | CtR2Up | CAATGCCAAATCGGCCACGGTTCCGAAAAATG | 79 |
| | CtR2Dw | ATCCGTAATATCCGCATTTTTCGGAAC | 80 |
| FpR1 | FpR1Up | ATGCAGAAAAAGTTAAA | 81 |
| | FpR1Dw | TTTAACTTTTTCTGCAT | 82 |
| FpR2 | FpR2Up | CGTCAAAAGCAAGCGTTGCAAAACATG | 83 |
| | FpR2Dw | TTCTTCCGGACTTGCATGTTTTGCAAC | 84 |
| CpR1 | CpR1f | TATGTCAAAGCAAAGGCAC | 85 |
| | CpR1r | TCAGAACATGTACCGGTACG | 86 |
| CpR2 | CpR2f | TACAGGTATGCTACCGGTTTTGAGTCTTTG | 87 |
| | CpR2r | CGTTCCTTCCCCTGCGGAC | 88 |
| FpR | FpR1f | TACGTTAGCGCCAAAG | 89 |
| | FpR1r | TTACCTGCGCTCAGAC | 90 |
| FpR2 | FpR2f | TATCGTGCAAGCACCGGTTTTG | 91 |
| | FpR2r | CGACCACGTTTAAAAACTGCCAGTTC | 92 |
| BsR1 | BsR1f | TATGTTTCAGCAAAGTCACA | 93 |
| | BsR1r | TCAGATCATTTGGTTTGGTAAAG | 94 |
| BsR2 | BsR2f | TACCGCTACAGTACAGC | 95 |
| | BsR2r | CGTTTCTGCCTCTTTTCAGC | 96 |
| SeR1 | SeR1f | TACGTTTCAGCGAAAAGTC | 97 |
| | SeR1r | TCAGACGATGAGGTTTACTTTGTAATTTTAG | 98 |
| SeR2 | SeR2f | TATCGTAAAAGTTCAGCGTTAG | 99 |
| | SeR2r | CGTTACGTCCTCGTTTTAAAAC | 100 |
| ET | ET-long | GTCCGGCGTAGAGGATCG | 101 |
| | ET-reverse | TCCCATTCGCCAATCC | 102 |

PCR was performed using Pfu polymerase. Reaction products were treated with phage T4 polynucleotide kinase in the presence of 1 mM ATP in order to phosphorylate DNA 5′-ends, and subsequently, they were combined with synthetic double-stranded oligonucleotides comprising a sequence encoding the exchanged element derived from a different microorganism (Table 10). In the case of sequences encoding α5b-α6 loop, the insert was obtained by filling in 3′-ends of the hybrid resulting from the renaturation of two oligonucleotides with partly complementary sequences (Table 11).

Ligation was conducted at room temperature for 1 hour in the presence of 5% PEG 4000 and 5 units of phage T4 DNA ligase. Ligation products were used to transform E. coli Top 10 strain, and then the selection was performed as described in Example 1. The material from single colonies was used in PCR using Taq polymerase and ET-long and ET-reverse primers (Table 12) which amplified the whole insert. Amplification product was subjected to sequencing which enabled the selection of clones with desired insert orientation in relation to vector sequence. Obtained chimeric proteins are listed in the table (Table 13).

TABLE 13
The origin of particular structural elements in produced chimeric proteins.

| | | | Donor | | |
|---|---|---|---|---|---|
| | | Donor of | of | Amino Acid | Nucleotide |
| Chimeric | | Transplantable | Transplantable | Sequence SEQ | Sequence |
| Protein | Acceptor Part | α4 Helix | α5b-α6 Loop | ID NO: | SEQ ID NO: |
| Ct(FpH) | CtMiniIII | FpMiniIII | — | 18 | 19 |
| Ct(FpL) | CtMiniIII | — | FpMiniIII | 20 | 21 |
| Ct(FpHL) | CtMiniIII | FpMiniIII | FpMiniIII | 22 | 23 |
| Bs(FpH) | BsMiniIII | FpMiniIII | — | 24 | 25 |
| Bs(FpL) | BsMiniIII | — | FpMiniIII | 26 | 27 |
| Se(FpH) | SeMiniIII | FpMiniIII | — | 28 | 29 |

In this way, the effective method for exchanging structural elements involved in the target sequence selection by Mini-III RNases has been developed and employed.

### Example 10

Expression and Purification of Mini-III RNase Protein Variants with Exchanged Structural Elements, and Analysis of the Sequence Preference Thereof

Overexpression and purification of Mini-III RNase chimeric proteins was performed as described in Example 2, wherein E. coli BL21(DE3) strain was transformed with plasmid carrying genes encoding Mini-III chimaeras. The next step was to measure the initial rate of cleaving the substrates selected from pUC910S substitution variants. The kinetics measurement for the cleavage of these substrates was performed as described in Example 8. The results are shown in FIG. 8. Enzymes Ct(FpH)MiniIII, Ct(FpL)MiniIII, Ct(FpHL)MiniIII, and Bs(FpH)MiniIII, demonstrated increased activity in relation to both initial enzymes forming the chimeric protein (CtMiniIII and FpMiniIII). Enzymes Ct(FpH)MiniIII, Ct(FpL)MiniIII, and Ct(FpHL)MiniIII showed significantly changed sequence preference. While 910S-UGCA substrate is cleaved by wild type CtMiniIII very slowly, chimeric protein Ct(FpL) cleaves this substrate much faster, and chimeric protein Ct(FpH) cleaves this substrate at a rate close to the rate for cleaving 910S-ACCU substrate. In the case of chimeric protein Ct(FpHL), 910S-UGCA substrate is cleaved with efficiency similar to the efficiency of FpMiniIII donor, almost three times faster than the original 910S-ACCU substrate (FIG. 8).

In this way, we have demonstrated the effectiveness of the method for exchanging transplantable α4 helix and/or transplantable α5b-α6 loop as a method for obtaining enzymes with changed/increased catalytic activity, and/or changed sequence preference.

### SEQUENCE LISTING

SEQ ID NO 1 amino acid sequence of BsMiniIII^{wt} RNase from Bacillus subtilis;
SEQ ID NO 2-amino acid sequence of CkMiniIII^{wt} RNase from Caldicellulosiruptor kristjanssonii;
SEQ ID NO 3-nucleotide sequence of CkMiniIII^{wt} RNase from Caldicellulosiruptor kristjanssonii;
SEQ ID NO 4-amino acid sequence of CrMiniIII^{wt} RNase from Clostridium ramosum;
SEQ ID NO 5-nucleotide sequence of CrMiniIII^{wt} RNase from Clostridium ramosum;
SEQ ID NO 6-amino acid sequence of CtMiniIII^{wt} RNase from Clostridium thermocellum;
SEQ ID NO 7-nucleotide sequence of CtMiniIII^{wt} RNase from Clostridium thermocellum;
SEQ ID NO 8-amino acid sequence of FpMiniIII^{wt} RNase from Faecalibacterium prausnitzii;
SEQ ID NO 9-nucleotide sequence of FpMiniIII^{wt} RNase from Faecalibacterium prausnitzii;
SEQ ID NO 10-amino acid sequence of FnMiniIII^{wt} RNase from Fusobacterium nucleatum subsp. Nucleaturn;
SEQ ID NO 11-nucleotide sequence of FnMiniIII^{wt} RNase from Fusobacterium nucleatum subsp. Nucleaturn;
SEQ ID NO 12-amino acid sequence of SeMiniIII^{wt} RNase from Staphylococcus epidermidis;
SEQ ID NO 13-nucleotide sequence of SeMiniIII^{wt} RNase from Staphylococcus epidermidis;
SEQ ID NO 14-amino acid sequence of TmMiniIII^{wt} RNase from Thermotoga maritima;
SEQ ID NO 15-nucleotide sequence of TmMiniIII^{wt} RNase from Thermotoga maritima;
SEQ ID NO 16-amino acid sequence of TtMiniIII^{wt} RNase from Thermoanaerobacter tengcongensis, presently Caldanaerobacter subterraneus subsp. Tengcongensis;
SEQ ID NO 17-nucleotide sequence of TtMiniIII^{wt} RNase from Thermoanaerobacter tengcongensis, presently Caldanaerobacter subterraneus subsp. Tengcongensis;
SEQ ID NO 18-amino acid sequence of chimeric protein-Ct(FpH);
SEQ ID NO 19-nucleotide sequence of chimeric protein-Ct(FpH);
SEQ ID NO 20-amino acid sequence of chimeric protein-Ct(FpL);
SEQ ID NO 21-nucleotide sequence of chimeric protein-Ct(FpL);
SEQ ID NO 22-amino acid sequence of chimeric protein-Ct(FpHL);
SEQ ID NO 23-nucleotide sequence of chimeric protein-Ct(FpHL);
SEQ ID NO 24-amino acid sequence of chimeric protein-Bs(FpH);
SEQ ID NO 25-nucleotide sequence of chimeric protein-Bs(FpH);
SEQ ID NO 26-amino acid sequence of chimeric protein-Bs(FpL);
SEQ ID NO 27-nucleotide sequence of chimeric protein-Bs(FpL);
SEQ ID NO 28-amino acid sequence of chimeric protein-Se(FpH);
SEQ ID NO 29-nucleotide sequence of chimeric protein-Se(FpH);
SEQ ID NO from 29 to 102-meaning provided in the description;
SEQ ID NO 103-nucleotide sequence of a fragment from one of the substrates (910S), being a fragment of bacteriophage phi6 (φ6) genome, within which dsRNA cleavage occurs.

SEQUENCE LISTING

| | |
|---|---|
| <110> | MIBMIK |
| <120> | Methods for changing sequence specificity of Mini-III RNases |
| <130> | PZ/3534/AGR/PCT |
| <160> | 103 |
| <170> | PatentIn version 3.5 |
| <210> | 1 |
| <211> | 143 |
| <212> | PRT |
| <213> | Bacillus subtilis |
| <400> | 1 |

```
Met Leu Glu Phe Asp Thr Ile Lys Asp Ser Lys Gln Leu Asn Gly Leu
1 5 10 15
Ala Leu Ala Tyr Ile Gly Asp Ala Ile Phe Glu Val Tyr Val Arg His
                        20 25 30
His Leu Leu Lys Gln Gly Phe Thr Lys Pro Asn Asp Leu His Lys Lys
                35 40 45
Ser Ser Arg Ile Val Ser Ala Lys Ser Gln Ala Glu Ile Leu Phe Phe
        50 55 60
Leu Gln Asn Gln Ser Phe Phe Thr Glu Glu Glu Glu Ala Val Leu Lys
65 70 75 80
Arg Gly Arg Asn Ala Lys Ser Gly Thr Thr Pro Lys Asn Thr Asp Val
                                85 90 95
Gln Thr Tyr Arg Tyr Ser Thr Ala Phe Glu Ala Leu Leu Gly Tyr Leu
                        100 105 110
Phe Leu Glu Lys Lys Glu Glu Arg Leu Ser Gln Leu Val Ala Glu Ala
                115 120 125
Ile Gln Phe Gly Thr Ser Gly Arg Lys Thr Asn Glu Ser Ala Thr
        130 135 140
```

| | |
|---|---|
| <210> | 2 |
| <211> | 132 |
| <212> | PRT |
| <213> | Caldicellulosiruptor kristjanssonii |
| <400> | 2 |

```
Met Leu Ser Pro Leu Val Tyr Ala Tyr Ile Gly Asp Ala Val Tyr Glu
1 5 10 15
Leu Phe Val Arg Asn Lys Ile Ile Ala Glu Asn Pro Asp Leu Thr Pro
                        20 25 30
Tyr Leu Tyr Tyr Leu Arg Thr Thr Met Tyr Val Lys Ala Ser Ser Gln
                35 40 45
Ala Met Ala Ile Lys Lys Leu Tyr Glu Glu Leu Asp Glu Asp Glu Lys
        50 55 60
Arg Ile Val Lys Arg Gly Arg Asn Ala Lys Pro Lys Thr Ile Pro Arg
65 70 75 80
Asn Ala Lys Leu Ser Asp Tyr Lys Tyr Ala Thr Ala Leu Glu Ala Leu
                                85 90 95
Ile Gly Tyr Leu Tyr Leu Ala Asn Asn Ile Glu Arg Leu Asn Tyr Ile
                        100 105 110
Leu Ser Gln Thr Tyr Asp Ile Ile Thr Glu Glu Tyr Ser Asn Ala Lys
                115 120 125
Asn Ser Cys Gln
        130
```

| | |
|---|---|
| <210> | 3 |
| <211> | 399 |
| <212> | DNA |
| <213> | Caldicellulosiruptor kristjanssonii |
| <400> | 3 |

| | |
|---|---|
| atgcttagtc ctttagtata tgcttatatt ggagatgcag tatatgagtt gtttgtaaga | 60 |
| aacaaaataa tagctgaaaa tccagatttg accccctacc tatactatct tagaactact | 120 |
| atgtatgtaa aagcttcgag tcaagcaatg gctataaaaa aattatatga agagcttgat | 180 |
| gaagatgaaa aaagaattgt aaagagaggc agaaatgcaa aaccaaaaac cattcccaga | 240 |
| aatgccaagt tgagtgatta taaatatgcc acggcccttg aggcactaat tggttatctt | 300 |
| tatttagcaa ataacattga gagattaaat tatattcttt cacaaacgta tgatataata | 360 |
| actgaagaat acagcaatgc caagaatagc tgtcaataa | 399 |

| | |
|---|---|
| <210> | 4 |
| <211> | 125 |
| <212> | PRT |
| <213> | Clostridium ramosum |
| <400> | 4 |

```
Met Gly Pro Glu Leu Ile Asn Ala Ser Val Leu Ala Tyr Leu Gly Asp
1 5 10 15
Ser Ile Phe Glu Val Leu Val Arg Asp Tyr Leu Val Lys Glu Ser Gly
                        20 25 30
Phe Val Lys Pro Asn Asp Leu Gln Arg Glu Ala Val Lys Tyr Val Ser
                35 40 45
Ala Ser Ser His Ala Ala Phe Met His Asp Met Leu Asp Glu Glu Phe
        50 55 60
Phe Ser Ala Asp Glu Val Gly Thr Tyr Lys Arg Gly Arg Asn Thr Lys
65 70 75 80
Gly Ser Lys Asn Glu Ser Leu Asp His Met His Ser Thr Gly Phe Glu
                                85 90 95
Ala Val Ile Gly Thr Leu Tyr Leu Glu Glu Asn Phe Asp Arg Ile Lys
                        100 105 110
Val Ile Phe Glu Arg Tyr Lys Gln Tyr Ile Asn Asn Lys
                115 120 125
```

| | |
|---|---|
| <210> | 5 |
| <211> | 378 |
| <212> | DNA |
| <213> | Clostridium ramosum |
| <400> | 5 |

| | |
|---|---|
| atgggccctg aactgattaa tgcaagcgtt ctggcatatc tgggtgatag catttttgaa | 60 |
| gttctggtgc gtgattatct ggtgaaagaa agcggttttg tgaaaccgaa tgatctgcag | 120 |
| cgtgaagccg ttaaatatgt tagcgcaagc agccatgcag catttatgca tgatatgctg | 180 |
| gatgaagaat ttttcagcgc agatgaagtt ggcacctata aacgtggtcg taataccaaa | 240 |
| ggtagcaaaa atgaaagcct ggatcatatg catagcaccg gttttgaagc agttattggc | 300 |
| accctgtatc tggaagaaaa tttcgatcgc atcaaagtga tcttcgagcg ctataaacag | 360 |
| tacatcaaca acaaataa | 378 |

| | |
|---|---|
| <210> | 6 |
| <211> | 140 |
| <212> | PRT |
| <213> | Clostridium thermocellum |
| <400> | 6 |

```
Met Val Trp Glu Phe Phe Asp Lys Ile Thr Gly Glu Phe Asn Tyr Lys
1 5 10 15
Pro Asp Glu Val Ser Gln Leu Ser Pro Leu Val Leu Ala Tyr Ile Gly
                        20 25 30
Asp Ala Val Tyr Glu Val Phe Ile Arg Thr Met Leu Val Ser Gly Gly
                35 40 45
Asn Val Pro Val His Val Leu His Lys Arg Ser Ile Ala Tyr Val Lys
        50 55 60
Ala Lys Ala Gln Ser Asp Ile Val His Arg Ile Met Pro Leu Leu Thr
65 70 75 80
Glu Glu Glu Leu Asn Ile Val Arg Arg Gly Arg Asn Ala Lys Ser Ala
                                85 90 95
Thr Val Pro Lys Asn Ala Asp Ile Thr Asp Tyr Arg Tyr Ala Thr Gly
                        100 105 110
Phe Glu Ser Leu Leu Gly Phe Leu Tyr Leu Lys Lys Asp Tyr Asp Arg
                115 120 125
Leu Met Asp Ile Leu Arg Met Ala Val Ser Gln Asn
        130 135 140
```

| | |
|---|---|
| <210> | 7 |
| <211> | 423 |
| <212> | DNA |
| <213> | Clostridium thermocellum |
| <400> | 7 |

| | |
|---|---|
| atggtttggg aattttttga caaaattaca ggtgagttta attacaaacc ggatgaagta | 60 |
| agccaactgt cgcctttagt gcttgcatac ataggtgacg ccgtgtatga ggttttcatc | 120 |
| cgtacaatgc ttgtgtccgg aggaaacgta ccggtacatg ttctccataa gcgctccatt | 180 |
| gcttatgtca aagcaaaggc acagtcggat attgtccaca ggataatgcc tttgctgacg | 240 |
| gaggaggagc ttaatattgt ccgcagggga aggaacgcca aatcggccac ggttccgaaa | 300 |
| aatgcggata ttacggatta caggtatgct accggttttg agtctttgtt gggttttctt | 360 |
| tatttgaaaa aagattatga ccgattgatg gatatattgc gaatggctgt ttcacagaat | 420 |
| tga | 423 |

| | |
|---|---|
| <210> | 8 |
| <211> | 134 |
| <212> | PRT |
| <213> | Faecalibacterium prausnitzii |
| <400> | 8 |

```
Met Asn Glu Ser Glu Lys Ile Asp Pro Arg Glu Leu Ser Pro Leu Ala
1 5 10 15
Leu Ala Phe Val Gly Asp Ser Val Leu Glu Leu Leu Val Arg Gln Arg
                        20 25 30
Leu Val Glu His His Arg Leu Ser Ala Gly Lys Leu Asn Ala Glu Lys
                35 40 45
Val Lys Tyr Val Ser Ala Lys Ala Gln Phe Arg Glu Glu Gln Leu Leu
        50 55 60
Glu Pro Leu Phe Thr Glu Asp Glu Leu Ala Val Phe Lys Arg Gly Arg
65 70 75 80
Asn Ala Ser Lys Ala Ser Val Ala Lys His Ala Ser Pro Glu Glu Tyr
                                85 90 95
Arg Ala Ser Thr Gly Phe Glu Cys Leu Leu Gly Trp Leu Tyr Leu Asn
                        100 105 110
Gly Gln Leu Glu Arg Val His Gln Leu Phe Glu Val Leu Trp Gln Gln
                115 120 125
Phe Asp Pro Asp Gln Lys
        130
```

| | |
|---|---|
| <210> | 9 |
| <211> | 405 |
| <212> | DNA |
| <213> | Faecalibacterium prausnitzii |
| <400> | 9 |

| | |
|---|---|
| atgaatgaaa gcgaaaaaat tgatccgcgt gaactgagtc cgctggcact ggcatttgtt | 60 |
| ggtgatagcg ttctggaact gctggttcgt cagcgtctgg ttgaacatca tcgtctgagc | 120 |
| gcaggtaaac tgaatgcaga aaaagttaaa tacgttagcg ccaaagcaca gtttcgtgaa | 180 |
| gaacagctgc tggaaccgct gtttaccgaa gatgaactgg cagtttttaa acgtggtcgt | 240 |
| aatgcaagca aagcaagcgt tgcaaaacat gcaagtccgg aagaatatcg tgcaagcacc | 300 |
| ggttttgaat gtctgctggg ttggctgtat ctgaatggtc agctggaacg tgttcatcag | 360 |
| ctgtttgaag ttctgtggca gcagtttgat cctgatcaga aataa | 405 |

| | |
|---|---|
| <210> | 10 |
| <211> | 129 |
| <212> | PRT |
| <213> | Fusobacterium nucleatum subsp. nucleatum |
| <400> | 10 |

```
Met Asp Asn Val Asp Phe Ser Lys Asp Ile Arg Asp Tyr Ser Gly Leu
1 5 10 15
Glu Leu Ala Phe Leu Gly Asp Ala Ile Trp Glu Leu Glu Ile Arg Lys
                        20 25 30
Tyr Tyr Leu Gln Phe Gly Tyr Asn Ile Pro Thr Leu Asn Lys Tyr Val
                35 40 45
Lys Ala Lys Val Asn Ala Lys Tyr Gln Ser Leu Ile Tyr Lys Lys Ile
        50 55 60
Ile Asn Asp Leu Asp Glu Glu Phe Lys Val Ile Gly Lys Arg Ala Lys
65 70 75 80
Asn Ser Asn Ile Lys Thr Phe Pro Arg Ser Cys Thr Val Met Glu Tyr
85 90 95
Lys Glu Ala Thr Ala Leu Glu Ala Ile Ile Gly Ala Met Tyr Leu Leu
                        100 105 110
Lys Lys Glu Glu Glu Ile Lys Lys Ile Ile Asn Ile Val Ile Lys Gly
115 120 125
Glu
```

| | |
|---|---|
| <210> | 11 |
| <211> | 390 |
| <212> | DNA |
| <213> | Fusobacterium nucleatum subsp. nucleatum |
| <400> | 11 |

| | |
|---|---|
| atggacaatg tagatttttc aaaggatata agagattaca gtggactgga attagcattt | 60 |
| ttaggagatg ctatttggga actggaaata agaaaatatt acttacaatt tggctataat | 120 |
| attcctactt taaataaata tgttaaagct aaggtaaatg caaaatatca aagtctgatt | 180 |
| tataagaaaa ttataaatga tttagatgaa gaatttaaag ttataggaaa aagagctaaa | 240 |
| aatagtaaca taaaaacttt tccaaggagt tgtacagtga tggaatataa ggaagcgaca | 300 |
| gccttagaag ctattatcgg agcaatgtat ttgttaaaaa aagaagaaga aataaaaaaa | 360 |
| attataaata tagttataaa gggagaatga | 390 |

| | |
|---|---|
| <210> | 12 |
| <211> | 132 |
| <212> | PRT |
| <213> | Staphylococcus epidermidis |
| <400> | 12 |

```
Met Ala Lys His Met Asn Val Lys Leu Leu Asn Pro Leu Thr Leu Ala
1 5 10 15
Tyr Met Gly Asp Ala Val Leu Asp Gln His Val Arg Glu Tyr Ile Val
                        20 25 30
Leu Lys Leu Gln Ser Lys Pro His Arg Leu His Gln Val Ser Lys Ser
                35 40 45
Tyr Val Ser Ala Lys Ser Gln Ala Lys Thr Leu Glu Tyr Leu Leu Asp
        50 55 60
Ile Asp Trp Phe Thr Glu Glu Glu Leu Ser Val Leu Lys Arg Gly Arg
65 70 75 80
Asn Ala Lys Ser Tyr Thr Lys Ala Lys Asn Thr Asp Ile Gln Thr Tyr
                                85 90 95
Arg Lys Ser Ser Ala Leu Glu Ala Val Ile Gly Phe Leu Tyr Leu Asp
                        100 105 110
His Gln Ser Glu Arg Leu Glu Asn Leu Leu Glu Thr Ile Val Arg Ile
                115 120 125
Val Asp Glu Arg
        130
```

| | |
|---|---|
| <210> | 13 |
| <211> | 399 |
| <212> | DNA |
| <213> | Staphylococcus epidermidis |
| <400> | 13 |

| | |
|---|---|
| atggctaaac atatgaacgt aaaacttctt aatcctttaa cattggcata tatgggtgat | 60 |
| gcagtacttg atcaacatgt gcgtgaatat atcgtgctaa aattacaaag taaacctcat | 120 |
| cgtttgcacc aagtatcgaa aagttacgtt tcagcgaaaa gtcaagctaa gactttagag | 180 |
| tatttgttag atattgactg gtttacagag gaagagctaa gtgttttaaa acgaggacgt | 240 |
| aacgctaaaa gttatacaaa agctaaaaat actgacattc aaacttatcg taaaagttca | 300 |
| gcgttagaag ctgttatcgg atttttatat ttagaccatc aatcagaacg attagaaaac | 360 |
| ttattagaaa caattgttag gatagtggat gaaaggtaa | 399 |

| | |
|---|---|
| <210> | 14 |
| <211> | 140 |
| <212> | PRT |
| <213> | Thermotoga maritima |
| <400> | 14 |

```
Met Glu Lys Leu Phe Arg Phe Glu Ala Glu Pro Glu Lys Leu Pro Pro
1 5 10 15
Ala Val Leu Ala Tyr Leu Gly Asp Ala Val Leu Glu Leu Ile Phe Arg
                        20 25 30
Ser Arg Phe Thr Gly Asp Tyr Arg Met Ser Val Ile His Glu Arg Val
                35 40 45
Lys Glu His Thr Ser Lys His Gly Gln Ala Trp Met Leu Glu Asn Ile
        50 55 60
Trp Asn Leu Leu Asp Glu Arg Glu Gln Glu Ile Val Lys Arg Ala Met
65 70 75 80
Asn Ser Lys Ala Ala Lys Arg His Gly Asn Asp Pro Thr Tyr Arg Lys
                                85 90 95
Ser Thr Gly Phe Glu Ala Leu Ile Gly Tyr Leu Phe Leu Lys Arg Glu
                        100 105 110
Phe Asp Arg Ile Glu Glu Leu Leu Arg Val Val Met Asp Leu Glu Ser
                115 120 125
Leu Arg Lys Lys Asn Pro Gly Gly Ser Ala Gln Glu
        130 135 140
```

| | |
|---|---|
| <210> | 15 |
| <211> | 423 |
| <212> | DNA |
| <213> | Thermotoga maritima |
| <400> | 15 |

| | |
|---|---|
| atggaaaaac tcttcagatt cgaagcagaa ccggagaaac tgccaccggc cgttctagcg | 60 |
| tatctgggag atgccgttct ggagctcatc ttcagatcga gattcacagg agattacaga | 120 |
| atgtccgtca tacacgagag ggtcaaggaa cacacctcga aacacggtca ggcatggatg | 180 |
| ctggagaata tatggaatct cctcgacgaa agagagcaag aaatagttaa aagagcgatg | 240 |
| aattcgaagg cagcgaaaag acacgggaac gaccctacat acagaaagag caccggtttc | 300 |
| gaagctttga tcgggtatct attcttgaaa agagaattcg acagaattga agaactgctt | 360 |
| cgggtggtga tggatcttga gagtctacgg aagaaaaatc ctggaggaag cgctcaggaa | 420 |
| taa | 423 |

| | |
|---|---|
| <210> | 16 |
| <211> | 136 |
| <212> | PRT |
| <213> | Thermoanaerobacter tengcongensis |
| <400> | 16 |

```
Met Glu Lys Asp Lys Met Ile Leu Val Lys Glu Lys Gly Val Leu Asp
1 5 10 15
Leu Ser Pro Leu Val Leu Ala Phe Ile Gly Asp Ala Val Tyr Ser Leu
                        20 25 30
Tyr Val Arg Thr Lys Ile Val Glu Lys Gly Asn Met Lys Leu Ala His
                35 40 45
Leu Asn Glu Gln Thr Val Lys Tyr Val Lys Ala Ser Ser Gln Ala Arg
        50 55 60
Ser Leu Glu Arg Ile Tyr Asp Leu Leu Thr Glu Glu Glu Lys Glu Ile
65 70 75 80
Val Arg Arg Gly Arg Asn Ala Lys Ala Ser Thr Val Pro Lys Gly Ala
                                85 90 95
Ser Val Lys Glu Tyr Lys Tyr Ala Thr Ala Phe Glu Ala Leu Val Gly
                        100 105 110
Tyr Leu Tyr Leu Leu Glu Arg Phe Asp Arg Leu Tyr Phe Leu Leu Ser
                115 120 125
Leu Ser Met Glu Tyr Thr Glu Glu
        130 135
```

| | |
|---|---|
| <210> | 17 |
| <211> | 477 |
| <212> | DNA |
| <213> | Thermoanaerobacter tengcongensis |
| <400> | 17 |

| | |
|---|---|
| atgggcagca gccatcatca tcatcatcac agcagcggcc tggaagttct gttccagggg | 60 |
| ccccatatgg aaaaggataa gatgattctt gtaaaggaaa agggggtttt agacttatcc | 120 |
| ccccttgttt tggctttcat tggagatgcg gtttacagcc tttatgtcag aactaagatt | 180 |
| gtggagaaag ggaatatgaa attggctcat ttaaatgagc aaactgtgaa gtacgttaag | 240 |
| gcatcttcac aggctaggtc tcttgagcga atttacgacc ttctcactga agaagaaaag | 300 |
| gaaattgtga gaaggggaag aaatgccaaa gcttctacag ttccaaaagg agcaagtgtt | 360 |
| aaagagtata agtatgccac tgcctttgaa gcattagtgg gatatttgta ccttttagaa | 420 |
| agatttgata ggctttactt tcttttgagc ctttctatgg aatacacgga agaatga | 477 |

| | |
|---|---|
| <210> | 18 |
| <211> | 140 |
| <212> | PRT |
| <213> | artificial |
| <220> | |
| <223> | AA sequence of chimeric protein Ct(FpH) |
| <400> | 18 |

```
Met Val Trp Glu Phe Phe Asp Lys Ile Thr Gly Glu Phe Asn Tyr Lys
1 5 10 15
Pro Asp Glu Val Ser Gln Leu Ser Pro Leu Val Leu Ala Tyr Ile Gly
                        20 25 30
Asp Ala Val Tyr Glu Val Phe Ile Arg Thr Met Leu Val Ser Gly Gly
                35 40 45
Asn Val Pro Val His Val Leu Asn Ala Glu Lys Val Lys Tyr Val Lys
        50 55 60
Ala Lys Ala Gln Ser Asp Ile Val His Arg Ile Met Pro Leu Leu Thr
65 70 75 80
Glu Glu Glu Leu Asn Ile Val Arg Arg Gly Arg Asn Ala Lys Ser Ala
                                85 90 95
Thr Val Pro Lys Asn Ala Asp Ile Thr Asp Tyr Arg Tyr Ala Thr Gly
                        100 105 110
Phe Glu Ser Leu Leu Gly Phe Leu Tyr Leu Lys Lys Asp Tyr Asp Arg
                115 120 125
Leu Met Asp Ile Leu Arg Met Ala Val Ser Gln Asn
        130 135 140
```

| | |
|---|---|
| <210> | 19 |
| <211> | 423 |
| <212> | DNA |
| <213> | artificial |
| <220> | |
| <223> | NA sequence of chimeric protein Ct(FpH) |
| <400> | 19 |

| | |
|---|---|
| atggtttggg aattttttga caaaattaca ggtgagttta attacaaacc ggatgaagta | 60 |
| agccaactgt cgcctttagt gcttgcatac ataggtgacg ccgtgtatga ggttttcatc | 120 |
| cgtacaatgc ttgtgtccgg aggaaacgta ccggtacatg ttctgaatgc agaaaaagtt | 180 |
| aaatatgtca aagcaaaggc acagtcggat attgtccaca ggataatgcc tttgctgacg | 240 |
| gaggaggagc ttaatattgt ccgcagggga aggaacgcca aatcggccac ggttccgaaa | 300 |
| aatgcggata ttacggatta caggtatgct accggttttg agtctttgtt gggttttctt | 360 |
| tatttgaaaa aagattatga ccgattgatg gatatattgc gaatggctgt ttcacagaat | 420 |
| taa | 423 |

| | |
|---|---|
| <210> | 20 |
| <211> | 140 |
| <212> | PRT |
| <213> | artificial |
| <220> | |
| <223> | AA sequence of chimeric protein Ct(FpL) |
| <400> | 20 |

```
Met Val Trp Glu Phe Phe Asp Lys Ile Thr Gly Glu Phe Asn Tyr Lys
1 5 10 15
Pro Asp Glu Val Ser Gln Leu Ser Pro Leu Val Leu Ala Tyr Ile Gly
                        20 25 30
Asp Ala Val Tyr Glu Val Phe Ile Arg Thr Met Leu Val Ser Gly Gly
                35 40 45
Asn Val Pro Val His Val Leu His Lys Arg Ser Ile Ala Tyr Val Lys
        50 55 60
Ala Lys Ala Gln Ser Asp Ile Val His Arg Ile Met Pro Leu Leu Thr
65 70 75 80
Glu Glu Glu Leu Asn Ile Val Arg Arg Gly Arg Asn Ala Ser Lys Ala
                                85 90 95
Ser Val Ala Lys His Ala Ser Pro Glu Glu Tyr Arg Tyr Ala Thr Gly
                        100 105 110
Phe Glu Ser Leu Leu Gly Phe Leu Tyr Leu Lys Lys Asp Tyr Asp Arg
                115 120 125
Leu Met Asp Ile Leu Arg Met Ala Val Ser Gln Asn
        130 135 140
```

| | |
|---|---|
| <210> | 21 |
| <211> | 423 |
| <212> | DNA |
| <213> | artificial |
| <220> | |
| <223> | NA sequence of chimeric protein Ct(FpL) |
| <400> | 21 |

| | |
|---|---|
| atggtttggg aattttttga caaaattaca ggtgagttta attacaaacc ggatgaagta | 60 |
| agccaactgt cgcctttagt gcttgcatac ataggtgacg ccgtgtatga ggttttcatc | 120 |
| cgtacaatgc ttgtgtccgg aggaaacgta ccggtacatg ttctccataa gcgctccatt | 180 |
| gcttatgtca aagcaaaggc acagtcggat attgtccaca ggataatgcc tttgctgacg | 240 |
| gaggaggagc ttaatattgt ccgcagggga aggaacgcgt caaaagcaag cgttgcaaaa | 300 |
| catgcaagtc cggaagaata caggtatgct accggttttg agtctttgtt gggttttctt | 360 |
| tatttgaaaa aagattatga ccgattgatg gatatattgc gaatggctgt ttcacagaat | 420 |
| taa | 423 |

| | |
|---|---|
| <210> | 22 |
| <211> | 140 |
| <212> | PRT |
| <213> | artificial |
| <220> | |
| <223> | AA sequence of chimeric protein Ct(FpHL) |
| <400> | 22 |

```
Met Val Trp Glu Phe Phe Asp Lys Ile Thr Gly Glu Phe Asn Tyr Lys
1 5 10 15
Pro Asp Glu Val Ser Gln Leu Ser Pro Leu Val Leu Ala Tyr Ile Gly
                        20 25 30
Asp Ala Val Tyr Glu Val Phe Ile Arg Thr Met Leu Val Ser Gly Gly
                35 40 45
Asn Val Pro Val His Val Leu Asn Ala Glu Lys Val Lys Tyr Val Lys
        50 55 60
Ala Lys Ala Gln Ser Asp Ile Val His Arg Ile Met Pro Leu Leu Thr
65 70 75 80
Glu Glu Glu Leu Asn Ile Val Arg Arg Gly Arg Asn Ala Ser Lys Ala
                                85 90 95
Ser Val Ala Lys His Ala Ser Pro Glu Glu Tyr Arg Tyr Ala Thr Gly
                        100 105 110
Phe Glu Ser Leu Leu Gly Phe Leu Tyr Leu Lys Lys Asp Tyr Asp Arg
                115 120 125
Leu Met Asp Ile Leu Arg Met Ala Val Ser Gln Asn
        130 135 140
```

| | |
|---|---|
| <210> | 23 |
| <211> | 422 |
| <212> | DNA |
| <213> | artificial |
| <220> | |
| <223> | NA sequence of chimeric protein Ct(FpHL) |
| <400> | 23 |

| | |
|---|---|
| atggtttggg aattttttga caaaattaca ggtgagttta attacaaacc ggatgaagta | 60 |
| agccaactgt cgcctttagt gcttgcatac ataggtgacg ccgtgtatga ggttttcatc | 120 |
| cgtacaatgc ttgtgtccgg aggaaacgta ccggtacatg ttctgaatgc agaaaaagtt | 180 |
| aaatatgtca aagcaaaggc acagtcggat attgtccaca ggataatgcc tttgctgacg | 240 |
| gaggaggagc ttaatattgt ccgcagggga aggaacgcgt caaaagcaag cgttgcaaaa | 300 |
| catgcaagtc cggaagaata caggtatgct accggttttg agtctttgtt gggttttctt | 360 |
| tatttgaaaa aagattatga ccgattgatg gatatattgc gaatggctgt ttcacagaat | 420 |
| ta | 422 |

| | |
|---|---|
| <210> | 24 |
| <211> | 141 |
| <212> | PRT |
| <213> | artificial |
| <220> | |
| <223> | AA sequence of chimeric protein Bs(FpH) |
| <400> | 24 |

```
Met Val Glu Phe Asp Thr Ile Lys Asp Ser Lys Gln Leu Asn Gly Leu
1 5 10 15
Ala Leu Ala Tyr Ile Gly Asp Ala Ile Phe Glu Val Tyr Val Arg His
                        20 25 30
His Leu Leu Lys Gln Gly Phe Thr Lys Pro Asn Asp Leu Asn Ala Glu
                35 40 45
Lys Tyr Val Ser Ala Lys Ser Gln Ala Glu Ile Leu Phe Phe Leu Gln
        50 55 60
Asn Gln Ser Phe Phe Thr Glu Glu Glu Glu Ala Val Leu Lys Arg Gly
65 70 75 80
Arg Asn Ala Lys Ser Gly Thr Thr Pro Lys Asn Thr Asp Val Gln Thr
                                85 90 95
Tyr Arg Tyr Ser Thr Ala Phe Glu Ala Leu Leu Gly Tyr Leu Phe Leu
                        100 105 110
Glu Lys Lys Glu Glu Arg Leu Ser Gln Leu Val Ala Glu Ala Ile Gln
                115 120 125
Phe Gly Thr Ser Gly Arg Lys Thr Asn Glu Ser Ala Thr
        130 135 140
```

| | |
|---|---|
| <210> | 25 |
| <211> | 426 |
| <212> | DNA |
| <213> | artificial |
| <220> | |
| <223> | NA sequence of chimeric protein Bs(FpH) |
| <400> | 25 |

| | |
|---|---|
| atggttgaat ttgatacgat aaaagattct aagcagctta acggtcttgc gcttgcttat | 60 |
| ataggtgatg ccatttttga agtgtatgtc aggcatcacc tgcttaagca gggctttacc | 120 |
| aaaccaaatg atctgaatgc agaaaaatat gtttcagcaa agtcacaggc tgagatccta | 180 |
| ttttttctgc agaatcaatc attttttacg gaagaagagg aagcggtgct gaaaagaggc | 240 |
| agaaatgcca agtcagggac aacacctaaa aatacagatg ttcagacgta ccgctacagt | 300 |
| acagcatttg aagcgcttct gggctacctt tttctagaga aaaaagagga acgacttagt | 360 |
| cagctcgtag ccgaagctat acaattcggg acgtcaggga ggaaaacaaa tgagtcagca | 420 |
| acataa | 426 |

| | |
|---|---|
| <210> | 26 |
| <211> | 143 |
| <212> | PRT |
| <213> | artificial |
| <220> | |
| <223> | AA sequence of chimeric protein Bs(FpL) |
| <400> | 26 |

```
Met Val Glu Phe Asp Thr Ile Lys Asp Ser Lys Gln Leu Asn Gly Leu
1 5 10 15
Ala Leu Ala Tyr Ile Gly Asp Ala Ile Phe Glu Val Tyr Val Arg His
                        20 25 30
His Leu Leu Lys Gln Gly Phe Thr Lys Pro Asn Asp Leu His Lys Lys
                35 40 45
Ser Ser Arg Ile Val Ser Ala Lys Ser Gln Ala Glu Ile Leu Phe Phe
        50 55 60
Leu Gln Asn Gln Ser Phe Phe Thr Glu Glu Glu Glu Ala Val Leu Lys
65 70 75 80
Arg Gly Arg Asn Ala Ser Lys Ala Ser Val Ala Lys His Ala Ser Pro
                                85 90 95
Glu Glu Tyr Arg Tyr Ser Thr Ala Phe Glu Ala Leu Leu Gly Tyr Leu
                        100 105 110
Phe Leu Glu Lys Lys Glu Glu Arg Leu Ser Gln Leu Val Ala Glu Ala
                115 120 125
Ile Gln Phe Gly Thr Ser Gly Arg Lys Thr Asn Glu Ser Ala Thr
        130 135 140
```

| | |
|---|---|
| <210> | 27 |
| <211> | 432 |
| <212> | DNA |
| <213> | artificial |
| <220> | |
| <223> | NA sequence of chimeric protein Bs(FpL) |
| <400> | 27 |

| | |
|---|---|
| atggttgaat ttgatacgat aaaagattct aagcagctta acggtcttgc gcttgcttat | 60 |
| ataggtgatg ccatttttga agtgtatgtc aggcatcacc tgcttaagca gggctttacc | 120 |
| aaaccaaatg atcttcataa gaaatcaagc cggattgttt cagcaaagtc acaggctgag | 180 |
| atcctatttt ttctgcagaa tcaatcattt tttacggaag aagaggaagc ggtgctgaaa | 240 |
| agaggcagaa acgcgtcaaa agcaagcgtt gcaaaacatg caagtccgga agaataccgc | 300 |
| tacagtacag catttgaagc gcttctgggc tacctttttc tagagaaaaa agaggaacga | 360 |
| cttagtcagc tcgtagccga agctatacaa ttcgggacgt cagggaggaa aacaaatgag | 420 |
| tcagcaacat aa | 432 |

| | |
|---|---|
| <210> | 28 |
| <211> | 131 |
| <212> | PRT |
| <213> | artificial |
| <220> | |
| <223> | AA sequence of chimeric protein Se(FpH) |
| <400> | 28 |

```
Met Val Ala Lys His Met Asn Val Lys Leu Leu Asn Pro Leu Thr Leu
1 5 10 15
Ala Tyr Met Gly Asp Ala Val Leu Asp Gln His Val Arg Glu Tyr Ile
                        20 25 30
Val Leu Lys Leu Gln Ser Lys Pro His Arg Leu Asn Ala Glu Lys Tyr
                35 40 45
Val Ser Ala Lys Ser Gln Ala Lys Thr Leu Glu Tyr Leu Leu Asp Ile
        50 55 60
Asp Trp Phe Thr Glu Glu Glu Leu Ser Val Leu Lys Arg Gly Arg Asn
65 70 75 80
Ala Lys Ser Tyr Thr Lys Ala Lys Asn Thr Asp Ile Gln Thr Tyr Arg
                                85 90 95
Lys Ser Ser Ala Leu Glu Ala Val Ile Gly Phe Leu Tyr Leu Asp His
                        100 105 110
Gln Ser Glu Arg Leu Glu Asn Leu Leu Glu Thr Ile Val Arg Ile Val
                115 120 125
Asp Glu Arg
        130
```

| | |
|---|---|
| <210> | 29 |
| <211> | 394 |
| <212> | DNA |
| <213> | artificial |
| <220> | |
| <223> | NA sequence of chimeric protein Se(FpH) |
| <400> | 29 |

| | |
|---|---|
| atggtggcta aacatatgaa cgtaaaactt cttaatcctt taacattggc atatatgggt | 60 |
| gatgcagtac ttgatcaaca tgtgcgtgaa tatatcgtgc taaaattaca aagtaaacct | 120 |
| catcgtctga atgcagaaaa atacgtttca gcgaaaagtc aagctaagac tttagagtat | 180 |
| ttgttagata ttgactggtt tacagaggaa gagctaagtg ttttaaaacg aggacgtaac | 240 |
| gctaaaagtt atacaaaagc taaaaatact gacattcaaa cttatcgtaa aagttcagcg | 300 |
| ttagaagctg ttatcggatt tttatattta gaccatcaat cagaacgatt agaaaactta | 360 |
| ttagaaacaa ttgttaggat agtggatgaa ataa | 394 |

| | |
|---|---|
| <210> | 30 |
| <211> | 27 |
| <212> | DNA |
| <213> | artificial |
| <220> | |
| <223> | Primer FckminiIII |
| <400> | 30 |

| | |
|---|---|
| cctccatggt cagtccttta gtatatg | 27 |

| | |
|---|---|
| <210> | 31 |
| <211> | 30 |
| <212> | DNA |
| <213> | artificial |
| <220> | |
| <223> | Primer RckminiIII |
| <400> | 31 |

| | |
|---|---|
| cctctcgagt tattgacagc tattcttggc | 30 |

| | |
|---|---|
| <210> | 32 |
| <211> | 28 |
| <212> | DNA |
| <213> | artificial |
| <220> | |
| <223> | Primer FcrminiIII |
| <400> | 32 |

| | |
|---|---|
| ggaccatggg ccctgaactg attaatgc | 28 |

| | |
|---|---|
| <210> | 33 |
| <211> | 30 |
| <212> | DNA |
| <213> | artificial |
| <220> | |
| <223> | Primer RcrminiIII |
| <400> | 33 |

| | |
|---|---|
| ggcctcgagt tatttgttgt tgatgtactg | 30 |

| | |
|---|---|
| <210> | 34 |
| <211> | 28 |
| <212> | DNA |
| <213> | artificial |
| <220> | |
| <223> | Primer FctminiIII |
| <400> | 34 |

| | |
|---|---|
| caggcatatg gtttgggaat tttttgac | 28 |

| | |
|---|---|
| <210> | 35 |
| <211> | 28 |
| <212> | DNA |
| <213> | artificial |
| <220> | |
| <223> | Primer RctminiIII |
| <400> | 35 |

| | |
|---|---|
| gacctcgagt caattctgtg aaacagcc | 28 |

| | |
|---|---|
| <210> | 36 |
| <211> | 27 |
| <212> | DNA |
| <213> | Artificial |
| <220> | |
| <223> | Primer FfpminiIII |
| <400> | 36 |

| | |
|---|---|
| ggaccatgga cgaaagcgaa aaaattg | 27 |

| | |
|---|---|
| <210> | 37 |
| <211> | 31 |
| <212> | DNA |
| <213> | Artificial |
| <220> | |
| <223> | Primer RfpminiIII |
| <400> | 37 |

| | |
|---|---|
| gcgctcgagt tatttctgat caggatcaaa c | 31 |

| | |
|---|---|
| <210> | 38 |
| <211> | 30 |
| <212> | DNA |
| <213> | artificial |
| <220> | |
| <223> | Primer FfnminiIII |
| <400> | 38 |

| | |
|---|---|
| ccgcatatgg acaatgtaga tttttcaaag | 30 |

| | |
|---|---|
| <210> | 39 |
| <211> | 54 |
| <212> | DNA |
| <213> | artificial |
| <220> | |
| <223> | Primer RfnminiIII |
| <400> | 39 |

| | |
|---|---|
| gtgctcgagt catcattctc cctttataac tatatttata atttttttta tttc | 54 |

| | |
|---|---|
| <210> | 40 |
| <211> | 31 |
| <212> | DNA |
| <213> | artificial |
| <220> | |
| <223> | Primer FseminiIII |
| <400> | 40 |

| | |
|---|---|
| tagacatatg gcagtggcta aacatatgaa c | 31 |

| | |
|---|---|
| <210> | 41 |
| <211> | 25 |
| <212> | DNA |
| <213> | artificial |
| <220> | |
| <223> | Primer RseminiIII |
| <400> | 41 |

| | |
|---|---|
| atctcgagct acctttcatc cacta | 25 |

| | |
|---|---|
| <210> | 42 |
| <211> | 29 |
| <212> | DNA |
| <213> | artificial |
| <220> | |
| <223> | Primer FtmminiIII |
| <400> | 42 |

| | |
|---|---|
| gcttcatatg gaaaaactct tcagattcg | 29 |

| | |
|---|---|
| <210> | 43 |
| <211> | 27 |
| <212> | DNA |
| <213> | artificial |
| <220> | |
| <223> | Primer RtmminiIII |
| <400> | 43 |

| | |
|---|---|
| cttctcgagt tattcctgag cgcttcc | 27 |

| | |
|---|---|
| <210> | 44 |
| <211> | 32 |
| <212> | DNA |
| <213> | artificial |
| <220> | |
| <223> | Primer FttminiIII |
| <400> | 44 |

| | |
|---|---|
| cgcacatatg gaaaaggata agatgattct tg | 32 |

| | |
|---|---|
| <210> | 45 |
| <211> | 32 |
| <212> | DNA |
| <213> | artificial |
| <220> | |
| <223> | Primer RttminiIII |
| <400> | 45 |

| | |
|---|---|
| gctctcgagt cattcttccg tgtattccat ag | 32 |

| | |
|---|---|
| <210> | 46 |
| <211> | 36 |
| <212> | DNA |
| <213> | artificial |
| <220> | |
| <223> | Primer UniShPreA |
| <400> | 46 |

| | |
|---|---|
| agatcggaag agcgtcgtgt agggaaagag tgtaga | 36 |

| | |
|---|---|
| <210> | 47 |
| <211> | 23 |
| <212> | DNA |
| <213> | artificial |
| <220> | |
| <223> | Primer UniShRT |
| <400> | 47 |

| | |
|---|---|
| tctacactct ttccctacac gac | 23 |

| | |
|---|---|
| <210> | 48 |
| <211> | 33 |
| <212> | DNA |
| <213> | artificial |
| <220> | |
| <223> | Primer PreA3Univ |
| <400> | 48 |

| | |
|---|---|
| gatcggaaga gcacacgtct gaactccagt cac | 33 |

| | |
|---|---|
| <210> | 49 |
| <211> | 34 |
| <212> | DNA |
| <213> | artificial |
| <220> | |
| <223> | Primer 910fT7 |
| <400> | 49 |

| | |
|---|---|
| taatacgact cactataggg ctgctcgcgc gttg | 34 |

| | |
|---|---|
| <210> | 50 |
| <211> | 31 |
| <212> | DNA |
| <213> | artificial |
| <220> | |
| <223> | Primer 910rP6 |
| <400> | 50 |

| | |
|---|---|
| ggaaaaaaat cagacacaac tgacgcgatc g | 31 |

| | |
|---|---|
| <210> | 51 |
| <211> | 41 |
| <212> | DNA |
| <213> | artificial |
| <220> | |
| <223> | Primer 949fT7 |
| <400> | 51 |

| | |
|---|---|
| taatacgact cactataggg cctctctctc tggccacgat c | 41 |

| | |
|---|---|
| <210> | 52 |
| <211> | 31 |
| <212> | DNA |
| <213> | artificial |
| <220> | |
| <223> | Primer 949rP6 |
| <400> | 52 |

| | |
|---|---|
| ggaaaaaaat gccctgtaca gcaggcataa g | 31 |

| | |
|---|---|
| <210> | 53 |
| <211> | 39 |
| <212> | DNA |
| <213> | artificial |
| <220> | |
| <223> | Primer 2021fT7 |
| <400> | 53 |

| | |
|---|---|
| taatacgact cactataggg ctcctatcat ggccgttgc | 39 |

| | |
|---|---|
| <210> | 54 |
| <211> | 30 |
| <212> | DNA |
| <213> | artificial |
| <220> | |
| <223> | Primer 2021rP6 |
| <400> | 54 |

| | |
|---|---|
| ggaaaaaaac ttcgagatca gggttggacg | 30 |

| | |
|---|---|
| <210> | 55 |
| <211> | 42 |
| <212> | DNA |
| <213> | artificial |
| <220> | |
| <223> | Primer 3292fT7 |
| <400> | 55 |

| | |
|---|---|
| taatacgact cactataggg taccgcgatc aacactgtcg tc | 42 |

| | |
|---|---|
| <210> | 56 |
| <211> | 29 |
| <212> | DNA |
| <213> | artificial |
| <220> | |
| <223> | Primer 3292rP6 |
| <400> | 56 |

| | |
|---|---|
| ggaaaaaaac gaatcaggac gtctggacg | 29 |

| | |
|---|---|
| <210> | 57 |
| <211> | 41 |
| <212> | DNA |
| <213> | artificial |
| <220> | |
| <223> | Primer 4486fT7 |
| <400> | 57 |

| | |
|---|---|
| taatacgact cactataggg ctgtctcccc tcggtttcat c | 41 |

| | |
|---|---|
| <210> | 58 |
| <211> | 28 |
| <212> | DNA |
| <213> | artificial |
| <220> | |
| <223> | Primer 4486rP6 |
| <400> | 58 |

| | |
|---|---|
| ggaaaaaaat cgacagacga cagcgctg | 28 |

| | |
|---|---|
| <210> | 59 |
| <211> | 42 |
| <212> | DNA |
| <213> | artificial |
| <220> | |
| <223> | Primer 4754fT7 |
| <400> | 59 |

| | |
|---|---|
| taatacgact cactataggg ctcatcgcct cgatgaacca ag | 42 |

| | |
|---|---|
| <210> | 60 |
| <211> | 29 |
| <212> | DNA |
| <213> | artificial |
| <220> | |
| <223> | Primer 4754rP6 |
| <400> | 60 |

| | |
|---|---|
| ggaaaaaaac tactgctttc gagcggtcg | 29 |

| | |
|---|---|
| <210> | 61 |
| <211> | 21 |
| <212> | DNA |
| <213> | artificial |
| <220> | |
| <223> | Primer WSSWf |
| <400> | 61 |

| | |
|---|---|
| sswctctctc tggccacgat c | 21 |

| | |
|---|---|
| <210> | 62 |
| <211> | 16 |
| <212> | DNA |
| <213> | artificial |
| <220> | |
| <223> | Primer WSSWr |
| <400> | 62 |

| | |
|---|---|
| wttccctccc agcacg | 16 |

| | |
|---|---|
| <210> | 63 |
| <211> | 21 |
| <212> | DNA |
| <213> | artificial |
| <220> | |
| <223> | Primer ANNTf |
| <220> | |
| <221> | misc_feature |
| <222> | (1)..(2) |
| <223> | n is a, c, g, or t |
| <400> | 63 |

| | |
|---|---|
| nntctctctc tggccacgat c | 21 |

| | |
|---|---|
| <210> | 64 |
| <211> | 16 |
| <212> | DNA |
| <213> | artificial |
| <220> | |
| <223> | Primer ANNTr |
| <400> | 64 |

| | |
|---|---|
| tttccctccc agcacg | 16 |

| | |
|---|---|
| <210> | 65 |
| <211> | 21 |
| <212> | DNA |
| <213> | artificial |
| <220> | |
| <223> | Primer NCCNf |
| <220> | |
| <221> | misc_feature |
| <222> | (3)..(3) |
| <223> | n is a, c, g, or t |
| <400> | 65 |

| | |
|---|---|
| ccnctctctc tggccacgat c | 21 |

| | |
|---|---|
| <210> | 66 |
| <211> | 16 |
| <212> | DNA |
| <213> | artificial |
| <220> | |
| <223> | Primer NCCNr |
| <220> | |
| <221> | misc_feature |
| <222> | (1)..(1) |
| <223> | n is a, c, g, or t |
| <400> | 66 |

| | |
|---|---|
| nttccctccc agcacg | 16 |

| | |
|---|---|
| <210> | 67 |
| <211> | 24 |
| <212> | DNA |
| <213> | artificial |
| <220> | |
| <223> | Primer 3T-r |
| <400> | 67 |

| | |
|---|---|
| tttacctccc agcacgaccg cgac | 24 |

| | |
|---|---|
| <210> | 68 |
| <211> | 26 |
| <212> | DNA |
| <213> | artificial |
| <220> | |
| <223> | Primer 3T-f |
| <400> | 68 |

| | |
|---|---|
| cctctctctc tggccacgat cgcgtc | 26 |

| | |
|---|---|
| <210> | 69 |
| <211> | 21 |
| <212> | DNA |
| <213> | artificial |
| <220> | |
| <223> | Primer 4C-r |
| <400> | 69 |

| | |
|---|---|
| gccctcccag cacgaccgcg a | 21 |

| | |
|---|---|
| <210> | 70 |
| <211> | 22 |
| <212> | DNA |
| <213> | artificial |
| <220> | |
| <223> | Primer 4G-r |
| <400> | 70 |

| | |
|---|---|
| cccctcccag cacgaccgcg ac | 22 |

| | |
|---|---|
| <210> | 71 |
| <211> | 19 |
| <212> | DNA |
| <213> | artificial |
| <220> | |
| <223> | Primer 4T-r |
| <400> | 71 |

| | |
|---|---|
| accctcccag cacgaccgc | 19 |

| | |
|---|---|
| <210> | 72 |
| <211> | 28 |
| <212> | DNA |
| <213> | artificial |
| <220> | |
| <223> | Primer 4-f |
| <400> | 72 |

| | |
|---|---|
| aacctctctc tctggccacg atcgcgtc | 28 |

| | |
|---|---|
| <210> | 73 |
| <211> | 28 |
| <212> | DNA |
| <213> | artificial |
| <220> | |
| <223> | Primer 11C-r |
| <400> | 73 |

| | |
|---|---|
| cctccctctc tggccacgat cgcgtcag | 28 |

| | |
|---|---|
| <210> | 74 |
| <211> | 28 |
| <212> | DNA |
| <213> | artificial |
| <220> | |
| <223> | Primer 11G-r |
| <400> | 74 |

| | |
|---|---|
| cctcgctctc tggccacgat cgcgtcag | 28 |

| | |
|---|---|
| <210> | 75 |
| <211> | 28 |
| <212> | DNA |
| <213> | artificial |
| <220> | |
| <223> | Primer 12A-r |
| <400> | 75 |

| | |
|---|---|
| cctctatctc tggccacgat cgcgtcag | 28 |

| | |
|---|---|
| <210> | 76 |
| <211> | 24 |
| <212> | DNA |
| <213> | artificial |
| <220> | |
| <223> | Primer 11/12-f |
| <400> | 76 |

| | |
|---|---|
| tttccctccc agcacgaccg cgac | 24 |

| | |
|---|---|
| <210> | 77 |
| <211> | 22 |
| <212> | DNA |
| <213> | artificial |
| <220> | |
| <223> | Primer CtR1Up |
| <400> | 77 |

| | |
|---|---|
| gcttcataag cgctccattg ct | 22 |

| | |
|---|---|
| <210> | 78 |
| <211> | 22 |
| <212> | DNA |
| <213> | artificial |
| <220> | |
| <223> | Primer CtR1Dw |
| <400> | 78 |

| | |
|---|---|
| agcaatggag cgcttatgaa gc | 22 |

| | |
|---|---|
| <210> | 79 |
| <211> | 32 |
| <212> | DNA |
| <213> | artificial |
| <220> | |
| <223> | Primer CtR2Up |
| <400> | 79 |

| | |
|---|---|
| caatgccaaa tcggccacgg ttccgaaaaa tg | 32 |

| | |
|---|---|
| <210> | 80 |
| <211> | 27 |
| <212> | DNA |
| <213> | artificial |
| <220> | |
| <223> | Primer CtR2Dw |
| <400> | 80 |

| | |
|---|---|
| atccgtaata tccgcatttt tcggaac | 27 |

| | |
|---|---|
| <210> | 81 |
| <211> | 17 |
| <212> | DNA |
| <213> | artificial |
| <220> | |
| <223> | Primer FpR1Up |
| <400> | 81 |

| | |
|---|---|
| atgcagaaaa agttaaa | 17 |

| | |
|---|---|
| <210> | 82 |
| <211> | 17 |
| <212> | DNA |
| <213> | artificial |
| <220> | |
| <223> | Primer FpR1Dw |
| <400> | 82 |

| | |
|---|---|
| tttaactttt tctgcat | 17 |

| | |
|---|---|
| <210> | 83 |
| <211> | 27 |
| <212> | DNA |
| <213> | artificial |
| <220> | |
| <223> | Primer FpR2Up |
| <400> | 83 |

| | |
|---|---|
| cgtcaaaagc aagcgttgca aaacatg | 27 |

| | |
|---|---|
| <210> | 84 |
| <211> | 27 |
| <212> | DNA |
| <213> | artificial |
| <220> | |
| <223> | Primer FpR2Dw |
| <400> | 84 |

| | |
|---|---|
| ttcttccgga cttgcatgtt ttgcaac | 27 |

| | |
|---|---|
| <210> | 85 |
| <211> | 19 |
| <212> | DNA |
| <213> | artificial |
| <220> | |
| <223> | Primer CpR1f |
| <400> | 85 |

| | |
|---|---|
| tatgtcaaag caaaggcac | 19 |

| | |
|---|---|
| <210> | 86 |
| <211> | 20 |
| <212> | DNA |
| <213> | artificial |
| <220> | |
| <223> | Primer CpR1r |
| <400> | 86 |

| | |
|---|---|
| tcagaacatg taccggtacg | 20 |

| | |
|---|---|
| <210> | 87 |
| <211> | 30 |
| <212> | DNA |
| <213> | artificial |
| <220> | |
| <223> | Primer CpR2f |
| <400> | 87 |

| | |
|---|---|
| tacaggtatg ctaccggttt tgagtctttg | 30 |

| | |
|---|---|
| <210> | 88 |
| <211> | 19 |
| <212> | DNA |
| <213> | artificial |
| <220> | |
| <223> | Primer |
| <400> | 88 |

| | |
|---|---|
| cgttccttcc cctgcggac | 19 |

| | |
|---|---|
| <210> | 89 |
| <211> | 16 |
| <212> | DNA |
| <213> | artificial |
| <220> | |
| <223> | Primer FpR1f |
| <400> | 89 |

| | |
|---|---|
| tacgttagcg ccaaag | 16 |

| | |
|---|---|
| <210> | 90 |
| <211> | 16 |
| <212> | DNA |
| <213> | artificial |
| <220> | |
| <223> | Primer FpR1r |
| <400> | 90 |

| | |
|---|---|
| ttacctgcgc tcagac | 16 |

| | |
|---|---|
| <210> | 91 |
| <211> | 22 |
| <212> | DNA |
| <213> | artificial |
| <220> | |
| <223> | Primer FpR2f |
| <400> | 91 |

| | |
|---|---|
| tatcgtgcaa gcaccggttt tg | 22 |

| | |
|---|---|
| <210> | 92 |
| <211> | 26 |
| <212> | DNA |
| <213> | artificial |
| <220> | |
| <223> | Primer FpR2r |
| <400> | 92 |

| | |
|---|---|
| cgaccacgtt taaaaactgc cagttc | 26 |

| | |
|---|---|
| <210> | 93 |
| <211> | 20 |
| <212> | DNA |
| <213> | artificial |
| <220> | |
| <223> | Primer BsR1f |
| <400> | 93 |

| | |
|---|---|
| tatgtttcag caaagtcaca | 20 |

| | |
|---|---|
| <210> | 94 |
| <211> | 23 |
| <212> | DNA |
| <213> | artificial |
| <220> | |
| <223> | Primer BsR1r |
| <400> | 94 |

| | |
|---|---|
| tcagatcatt tggtttggta aag | 23 |

| | |
|---|---|
| <210> | 95 |
| <211> | 17 |
| <212> | DNA |
| <213> | artificial |
| <220> | |
| <223> | Primer BsR2f |
| <400> | 95 |

| | |
|---|---|
| taccgctaca gtacagc | 17 |

| | |
|---|---|
| <210> | 96 |
| <211> | 20 |
| <212> | DNA |
| <213> | artificial |
| <220> | |
| <223> | Primer BsR2r |
| <400> | 96 |

| | |
|---|---|
| cgtttctgcc tcttttcagc | 20 |

| | |
|---|---|
| <210> | 97 |
| <211> | 19 |
| <212> | DNA |
| <213> | artificial |
| <220> | |
| <223> | Primer SeR1f |
| <400> | 97 |

| | |
|---|---|
| tacgtttcag cgaaaagtc | 19 |

| | |
|---|---|
| <210> | 98 |
| <211> | 31 |
| <212> | DNA |
| <213> | artificial |
| <220> | |
| <223> | Primer SeR1rf |
| <400> | 98 |

| | |
|---|---|
| tcagacgatg aggtttactt tgtaatttta g | 31 |

| | |
|---|---|
| <210> | 99 |
| <211> | 22 |
| <212> | DNA |
| <213> | artificial |
| <220> | |
| <223> | Primer SeR2f |
| <400> | 99 |

| | |
|---|---|
| tatcgtaaaa gttcagcgtt ag | 22 |

| | |
|---|---|
| <210> | 100 |
| <211> | 22 |
| <212> | DNA |
| <213> | artificial |
| <220> | |
| <223> | Primer SeR2r |
| <400> | 100 |

| | |
|---|---|
| cgttacgtcc tcgttttaaa ac | 22 |

| | |
|---|---|
| <210> | 101 |
| <211> | 18 |
| <212> | DNA |
| <213> | artificial |
| <220> | |
| <223> | Primer ET-long |
| <400> | 101 |

| | |
|---|---|
| gtccggcgta gaggatcg | 18 |

| | |
|---|---|
| <210> | 102 |
| <211> | 16 |
| <212> | DNA |
| <213> | artificial |
| <220> | |
| <223> | Primer ET-reverse |
| <400> | 102 |

| | |
|---|---|
| tcccattcgc caatcc | 16 |

| | |
|---|---|
| <210> | 103 |
| <211> | 14 |
| <212> | RNA |
| <213> | phi6 bacteriophage |
| <400> | 103 |

| | |
|---|---|
| gggaaaccuc ucuc | 14 |

1. A Mini-III RNase with amino acid sequence comprising an acceptor part, and a transplantable α4 helix, and a transplantable α5b-α6 loop, which form structures of α4 helix and α5b-α6 loop, respectively, in the Mini-III RNase structure,
wherein the fragments which form structures of α4 helix and α5b-α6 loop, respectively, correspond structurally to respective structures of α4 helix and α5b-α6 loop formed by amino acid sequence fragments 46-52 and 85-98, respectively, of Mini-III RNase from Bacillus stubtilis shown in SEQ ID NO: 1,
wherein said Mini-III RNase exhibits sequence specificity in dsRNA cleavage that is dependent only on a ribonucleotide sequence of a substrate, and independent from an occurrence of secondary structures in the substrate's structure, and independent from a presence of other assisting proteins, and wherein the Mini-III RNase is not the Mini-III protein from Bacillus stubtilis of SEQ ID NO: 1 , nor SEQ ID NO: 1 with D94R mutation.

2. The Mini-III RNase according to claim 1, characterised in that the amino acid sequence is constructed of an acceptor part, derived from a Mini-III RNase of one microorganism, with inserted transplantable α4 helix and/or transplantable α5b-α6 loop, respectively, derived from α4 helix and/or α5b-α6 loop sequence of a Mini-III RNase from a different microorganism.

3. The Mini-III RNase according to claim 1 or 2, characterised in that the amino acid sequence thereof includes
an acceptor part derived from Mini-III RNase of BsMiniIII^{wt} (SEQ ID NO: 1), or Mini-III CkMiniIII^{wt} of Caldicellulosiruptor kristjanssonii (SEQ ID NO: 2), or CrMiniIII^{wt} of Clostridium ramosum (SEQ ID NO: 4), or CtMiniIII^{wt} of Clostridium thermocellum (SEQ ID NO: 6), or FpMiniIII^{wt} of Faecalibacterium prausnitzii (SEQ ID NO: 8), or FnMiniIII^{wt} of Fusobacterium nucleatum subsp. nucleatum (SEQ ID NO: 10), or SeMiniIII of Staphylococcus epidermidis (SEQ ID NO: 12), or TmMiniIII^{wt} of Thermotoga maritima (SEQ ID NO: 14), or TtMiniIII^{wt} of Thermoanaerobacter tengcongensis, presently Caldanaerobacter subterraneus subsp. tengcongensis (SEQ ID NO: 16) or an amino acid sequence identical therewith in at least 80%, preferably in 85%, more preferably in 90%, most preferably in 95%;
a transplantable α4 helix derived from
BsMiniIII^{wt} with amino acid sequence including amino acids in positions 46-52 of SEQ ID NO: 1, or from CkMiniIII^{wt} with amino acid sequence including amino acids 36-42 of SEQ ID NO: 2, or from CtMiniIII^{wt} with amino acid sequence including amino acids 40-46 of SEQ ID NO: 4, or from CtMiniIII^{wt} with amino acid sequence including amino acids in positions 56-62 of SEQ ID NO: 6, or from FpMiniIII^{wt} with amino acid sequence including amino acids in positions 45-51 of SEQ ID NO: 8, or from FnMiniIII^{wt} with amino acid sequence including amino acids 45-51 of SEQ ID NO: 10, or from SeMiniIII^{wt} with amino acid sequence including amino acids in positions 43-49 of SEQ ID NO: 12, or from TmMiniIII^{wt} with amino acid sequence including amino acids 45-51 of SEQ ID NO: 14, or from TtMiniIII^{wt} with amino acid sequence including amino acids 50-56 of SEQ ID NO: 16) or an amino acid sequence identical therewith in at least 80%, preferably in 85%, more preferably in 90%, most preferably in 95%, and/or
a transplantable α5b-α6 loop derived from
BsMiniIII^{wt} with amino acid sequence including amino acids in positions 85-98 of SEQ ID NO: 1, or from CkMiniIII^{wt} with amino acid sequence including amino acids 73-86 of SEQ ID NO: 2, or from CtMiniIII^{wt} with amino acid sequence including amino acids 79-88 of SEQ ID NO: 4, or from CtMiniIII^{wt} with amino acid sequence including amino acids in positions 93-106 of SEQ ID NO: 6, or from FpMiniIII^{wt} with amino acid sequence including amino acids in positions 82-95 of SEQ ID NO: 8, or from FnMiniIII^{wt} with amino acid sequence including amino acids 82-95 of SEQ ID NO: 10, or from SeMiniIII^{wt} with amino acid sequence including amino acids in positions 82-95 of SEQ ID NO: 12, or from TmMiniIII^{wt} with amino acid sequence including amino acids 82-93 of SEQ ID NO: 14, or from TtMiniIII^{wt} with amino acid sequence including amino acids 87-100 of SEQ ID NO: 16, or an amino acid sequence identical therewith in at least 80%, preferably in 85%, more preferably in 90%, most preferably in 95%.

4. The Mini-III RNase according to claim 1, characterised in that it maintains the Mini-III RNase activity and includes a sequence or a fragment of an amino acid sequence from Caldicellulosiruptor kristjanssonii shown in SEQ ID NO: 2, wherein the Mini-III RNase shows sequence specificity in dsRNA cleavage and cleaves dsRNA within a consensus sequence
↓
WSSWNNYY
WSSWNNRR
↑
where N=A, C, G, U; W=A, U; S=C, G; Y=C, U.

5. The Mini-III RNase according to claim 1, characterised in that it maintains the Mini-III RNase activity and includes a sequence or a fragment of an amino acid sequence from Clostridium ramosum shown in SEQ ID NO: 4, wherein the Mini-III RNase shows sequence specificity in dsRNA cleavage and cleaves dsRNA within a consensus sequence
```
        ↓
SNWSSW
SNWSSW
    ↑
```
where N=A, C, G, U; W=A, U; S=C, G.

6. The Mini-III RNase according to claim 1, characterised in that it maintains the Mini-III RNase activity and includes a sequence or a fragment of an amino acid sequence from Clostridium thermocellum shown in SEQ ID NO: 6, wherein the Mini-III RNase shows sequence specificity in dsRNA cleavage and cleaves dsRNA within a consensus sequence
↓
WSSW
WSSW
↑
where W=A, U; S=C, G.

7. The Mini-III RNase according to claim 1, characterised in that it maintains the Mini-III RNase activity and includes a sequence or a fragment of an amino acid sequence from Faecalibacterium prausnitzii shown in SEQ ID NO: 8, wherein the Mini-III RNase shows sequence specificity in dsRNA cleavage and cleaves dsRNA within a consensus sequence
↓
WSSW
WSSW
↑
where W=A, U; S=C, G.

8. The Mini-III RNase according to claim 1, characterised in that it maintains the Mini-III RNase activity and includes a sequence or a fragment of an amino acid sequence from Fusobacterium nucleatum shown in SEQ ID NO: 10, wherein the Mini-III RNase shows sequence specificity in dsRNA cleavage and cleaves dsRNA within a consensus sequence
↓
ASSW
USSW
↑
where W=A, U; S=C, G.

9. The Mini-III RNase according to claim 1, characterised in that it maintains the Mini-III RNase activity and includes a sequence or a fragment of an amino acid sequence from Staphylococcus epidermidis shown in SEQ ID NO: 12, wherein the Mini-III RNase shows sequence specificity in dsRNA cleavage and cleaves dsRNA within a consensus sequence
↓
WNSU
WNSA
↑
where N=A, C, G, U; W=A, U; S=C, G.

10. The Mini-III RNase according to claim 1, characterised in that it maintains the Mini-III RNase activity and includes a sequence or a fragment of an amino acid sequence from Thermotoga maritima shown in SEQ ID NO: 14, wherein the Mini-III RNase shows sequence specificity in dsRNA cleavage and cleaves dsRNA within a consensus sequence
↓
WSSWNG
WSSWNC
↑
where N=A, C, G, U; W=A, U; S=C, G.

11. The Mini-III RNase according to claim 1, characterised in that it maintains the Mini-III RNase activity and includes a sequence or a fragment of an amino acid sequence from Thermoanaerobacter tengcongensis (Caldanaerobacter subterraneus subsp. tengcongensis) shown in SEQ ID NO: 16, wherein the Mini-III RNase shows sequence specificity in dsRNA cleavage and cleaves dsRNA within a consensus sequence
↓
WSSWNNYY
WSSWNNYY
↑
where N=A, C, G, U; W=A, U; S=C, G; Y=C, U.

12. The Mini-III RNase according to claims 1-3, characterised in that the Mini-III RNase is a chimeric protein selected from Ct(FpH) of SEQ ID NO: 18, Ct(FpL) of SEQ ID NO: 20, Ct(FpHL) of SEQ ID NO: 22, Bs(FpH) of SEQ ID NO: 24, Bs(FpL) of SEQ ID NO: 26, Se(FpH) of SEQ ID NO: 28.

13. A method of obtaining a chimeric Mini-III RNase, characterised in that the method includes the steps of:
a) cloning a gene which encodes the Mini-III RNase, wherein amino acid sequence thereof includes fragments which form structures of α4 helix and α5b-α6 loop, respectively, corresponding structurally to respective structures of α4 helix and α5b-α6 loop formed by amino acid sequence fragments 46-52 and 85-98, respectively, of Mini-III RNase from Bacillus stubtilis shown in SEQ ID NO: 1,
b) modifying the gene which encodes said RNase by exchanging at least one of the fragments encoding α4 helix and/or α5b-α6 loop structures, respectively, with a fragment encoding α4 helix and/or α5b-α6 loop structures, respectively, from a gene which encodes a Mini-III RNase of a different microorganism,
wherein said Mini-III RNase shows sequence specificity in dsRNA cleavage being dependent only on aribonucleotide sequence and independent from an occurrence of secondary structures in the substrate's structure, and independent from a presence of other assisting proteins,
and wherein the Mini-III RNase is not the Mini-III protein from Bacillus stubtilis with amino acid sequence shown in SEQ ID NO: 1, nor SEQ ID NO: 1 with D94R mutation.

14. The method of obtaining a chimeric Mini-III RNase according to claim 13, characterised in that step b) involves an insertion of a transplantable α4 helix and/or a transplantable α5b-α6 loop into an acceptor part, wherein
the acceptor part is derived from Mini-III RNase of BsMiniIII^{wt} (SEQ ID NO: 1), or Mini-III CkMiniIII^{wt} of Caldicellulosiruptor kristjanssonii (SEQ ID NO: 2), or CrMiniIII^{wt} of Clostridium ramosum (SEQ ID NO: 4), or CtMiniIII^{wt} of Clostridium thermocellum (SEQ ID NO: 6), or FpMiniIII^{wt} of Faecalibacterium prausnitzii (SEQ ID NO: 8), or FnMiniIII^{wt} of Fusobacterium nucleatum subsp. nucleatum (SEQ ID NO: 10), or SeMiniIII^{wt} of Staphylococcus epidermidis (SEQ ID NO: 12), or TmMiniIII^{wt} of Thermotoga maritima (SEQ ID NO: 14), or TtMiniIII^{wt} of Thermoanaerobacter tengcongensis, presently Caldanaerobacter subterraneus subsp. tengcongensis (SEQ ID NO: 16), or includes an amino acid sequence identical therewith in at least 80%, preferably in 85%, more preferably in 90%, most preferably in 95%;
the transplantable α4 helix is derived from
BsMiniIII^{wt} with amino acid sequence including amino acids in positions 46-52 of SEQ ID NO: 1, or from CkMiniIII^{wt} with amino acid sequence including amino acids 36-42 of SEQ ID NO: 2, or from CtMiniIII^{wt} with amino acid sequence including amino acids 40-46 of SEQ ID NO: 4, or from CtMiniIII^{wt} with amino acid sequence including amino acids in positions 56-62 of SEQ ID NO: 6, or from FpMiniIII^{wt} with amino acid sequence including amino acids in positions 45-51 of SEQ ID NO: 8, or from FnMiniIII^{wt} with amino acid sequence including amino acids 45-51 of SEQ ID NO: 10, or from SeMiniIII^{wt} with amino acid sequence including amino acids in positions 43-49 of SEQ ID NO: 12, or from TmMiniIII^{wt} with amino acid sequence including amino acids 45-51 of SEQ ID NO: 14, or from TtMiniIII^{wt} with amino acid sequence including amino acids 50-56 of SEQ ID NO: 16, or includes an amino acid sequence identical therewith in at least 80%, preferably in 85%, more preferably in 90%, most preferably in 95%, and/or
the transplantable α5b-α6 loop is derived from
BsMiniIII^{wt} with amino acid sequence including amino acids in positions 85-98 of SEQ ID NO: 1, or from CkMiniIII^{wt} with amino acid sequence including amino acids 73-86 of SEQ ID NO: 2, or from CtMiniIII^{wt} with amino acid sequence including amino acids 79-88 of SEQ ID NO: 4, or from CtMiniIII^{wt} with amino acid sequence including amino acids in positions 93-106 of SEQ ID NO: 6, or from FpMiniIII^{wt} with amino acid sequence including amino acids in positions 82-95 of SEQ ID NO: 8, or from FnMiniIII' with amino acid sequence including amino acids 82-95 of SEQ ID NO: 10, or from SeMiniIII^{wt} with amino acid sequence including amino acids in positions 82-95 of SEQ ID NO: 12, or from TmMiniIII^{wt} with amino acid sequence including amino acids 82-93 of SEQ ID NO: 14, or from TtMiniIII^{wt} with amino acid sequence including amino acids 87-100 of SEQ ID NO: 16, or includes an amino acid sequence identical therewith in at least 80%, preferably in 85%, more preferably in 90%, most preferably in 95%.

15. The method of obtaining a chimeric Mini-III RNase according to claims 13-14, characterised in that the transplantable α4 helix and the transplantable α5b-α6 loop in the gene encoding Mini-III RNase are derived from different microorganisms.

16. The method of obtaining a chimeric Mini-III RNase according to claims 13-15, characterised in that the gene encoding Mini-III RNase comprises any sequence which encodes an amino acid sequence from a group consisting of SEQ ID NO: 18, 20, 22, 24, 26, 28.

17. The method of obtaining a chimeric Mini-III RNase according to claims 13-16, characterised in that the method further includes the steps of
c) culturing cells which express the gene from step b), and
d) isolating and purifying the expressed protein from step c), and optionally step
e) of determining the sequence specificity of the protein obtained in step d).

18. A Mini-III RNase obtained with the method according to claims 13-17.

19. A construct encoding the Mini-III RNase according to claims 1-12, 18.

20. A cell comprising the gene encoding the Mini-III RNase according to claims 1-12, 18, or the construct according to claim 19.

21. Use of the Mini-III RNase according to claims 1-12 and 18 to cleave dsRNA in a manner dependent only on a ribonucleotide sequence, and independent from an occurrence of secondary structures in the substrate's structure, and independent from a presence of other assisting proteins.

22. A method of cleaving dsRNA in a manner dependent only on a ribonucleotide sequence, and independent from an occurrence of secondary structures in the substrate's structure, and independent from an presence of other assisting proteins, characterised in that the method includes interaction between a dsRNA substrate and the Mini-III RNase according to claims 1-12 or claim 18.

23. The method of cleaving dsRNA according to claim 22, characterised in that the Mini-III RNase includes a sequence from Caldicellulosiruptor kristjanssonii shown in SEQ ID NO: 2, wherein the Mini-III RNase shows sequence specificity in dsRNA cleavage and cleaves dsRNA within a consensus sequence
↓
WSSWNNYY
WSSWNNRR
↑
where N=A, C, G, U; W=A, U; S=C, G; Y=C, U.

24. The method of cleaving dsRNA according to claim 22, characterised in that the Mini-III RNase includes a sequence from Clostridium ramosum shown in SEQ ID NO: 4, wherein the Mini-III RNase shows sequence specificity in dsRNA cleavage and cleaves dsRNA within a consensus sequence
```
        ↓
SNWSSW
SNWSSW
    ↑
```
where N=A, C, G, U; W=A, U; S=C, G.

25. The method of cleaving dsRNA according to claim 22, characterised in that the Mini-III RNase includes a sequence from Clostridium thermocellum shown in SEQ ID NO: 6, wherein the Mini-III RNase shows sequence specificity in dsRNA cleavage and cleaves dsRNA within a consensus sequence
↓
WSSW
WSSW
↑
where W=A, U; S=C, G.

26. The method of cleaving dsRNA according to claim 22, characterised in that the Mini-III RNase includes a sequence from Faecalibacterium prausnitzii shown in SEQ ID NO: 8, wherein the Mini-III RNase shows sequence specificity in dsRNA cleavage and cleaves dsRNA within a consensus sequence
↓
WSSW
WSSW
↑
where W=A, U; S=C, G.

27. The method of cleaving dsRNA according to claim 22, characterised in that the Mini-III RNase includes a sequence from Fusobacterium nucleatum shown in SEQ ID NO: 10, wherein the Mini-III RNase shows sequence specificity in dsRNA cleavage and cleaves dsRNA within a consensus sequence
↓
ASSW
USSW
↑
where W=A, U; S=C, G.

28. The method of cleaving dsRNA according to claim 22, characterised in that the Mini-III RNase includes a sequence from Staphylococcus epidermidis shown in SEQ ID NO: 12, wherein the Mini-III RNase shows sequence specificity in dsRNA cleavage and cleaves dsRNA within a consensus sequence
↓
WNSU
WNSA
↑
where N=A, C, G, U; W=A, U; S=C, G.

29. The method of cleaving dsRNA according to claim 22, characterised in that the Mini-III RNase includes a sequence from Thermotoga maritima shown in SEQ ID NO: 14, wherein the Mini-III RNase shows sequence specificity in dsRNA cleavage and cleaves dsRNA within a consensus sequence
↓
WSSWNG
WSSWNC
↑
where N=A, C, G, U; W=A, U; S=C, G.

30. The method of cleaving dsRNA according to claim 22, characterised in that the Mini-III RNase includes a sequence from Thermoanaerobacter tengcongensis (Caldanaerobacter subterraneus subsp. tengcongensis) shown in SEQ ID NO: 16, wherein the Mini-III RNase shows sequence specificity in dsRNA cleavage and cleaves dsRNA within a consensus sequence
↓
WSSWNNYY
WSSWNNYY
↑
where N=A, C, G, U; W=A, U; S=C, G; Y=C, U.
